(19) United States
(12) Patent Application Publication
(10) Pub. No.: US 2009/0156562 A1
(43) Pub. Date: Jun. 18, 2009

(54) NOVEL COLORED SOLUTIONS OF INJECTABLE DRUGS AND THEIR PHARMACEUTICALLY ACCEPTABLE SALTS

(76) Inventor: Peter D. Winch, New Albany, OH (US)

Correspondence Address: BANNER & WITCOFF, LTD., 1100 13th STREET, N.W., SUITE 1200, WASHINGTON, DC, 20005-4051, US

(21) Appl. No.: 12/093,534

(22) PCT Filed: Nov. 13, 2006
(86) PCT No.: PCT/US06/43963

§ 371 Date: Nov. 18, 2008

Related U.S. Application Data
(60) Provisional application No. 60/735,578, filed on Nov. 10, 2005.
(60) Provisional application No. 60/736,577, filed on Nov. 14, 2005.
(60) Provisional application No. 60/736,579, filed on Nov. 14, 2005.
(60) Provisional application No. 60/736,575, filed on Nov. 14, 2005.
(60) Provisional application No. 60/736,573, filed on Nov. 14, 2005.
(60) Provisional application No. 60/736,574, filed on Nov. 14, 2005.
(60) Provisional application No. 60/736,372, filed on Nov. 14, 2005.
(60) Provisional application No. 60/736,373, filed on Nov. 14, 2005.
(60) Provisional application No. 60/735,370, filed on Nov. 12, 2005.
(60) Provisional application No. 60/736,374, filed on Nov. 14, 2005.
(60) Provisional application No. 60/736,468, filed on Nov. 14, 2005.
(60) Provisional application No. 60/736,464, filed on Nov. 14, 2005.
(60) Provisional application No. 60/761,282, filed on Jan. 23, 2006.
(60) Provisional application No. 60/761,283, filed on Jan. 23, 2006.
(60) Provisional application No. 60/761,277, filed on Jan. 23, 2006.
(60) Provisional application No. 60/761,274, filed on Jan. 23, 2006.
(60) Provisional application No. 60/761,276, filed on Jan. 23, 2006.

(51) Int. Cl. A61K 31/661; A61K 31/4709; A61K 31/58; A61K 31/5517; A61K 31/55; A61K 31/4748; A61K 31/47; A61K 31/5513; A61K 31/4164; A61K 31/40; A61K 31/225; A61K 31/135; A61K 31/05

(52) U.S. Cl. 514/130; 514/314; 514/176; 514/220; 514/221; 514/304; 514/308; 514/400; 514/424; 514/547; 514/647; 514/731

(57) ABSTRACT

The invention is directed to pharmaceutical compositions comprising colored solutions, colored emulsions, or colored powders of injectable pharmaceuticals wherein said pharmaceuticals are selected from the group consisting of muscle relaxants, hypnotics, induction agents, and anticholinergics. The formulations of the present invention may all be colored using fluorescein. Different colors may be achieved by either varying the concentration of fluorescein, or by combining fluorescein with another dye. The invention is also directed to methods involving the use of said pharmaceutical compositions.

### FIELD OF THE INVENTION

The present invention relates to compositions and methods comprising colored solutions of injectable drugs and their pharmaceutically acceptable salts.

### BACKGROUND

The safety of drug delivery is of critical importance to the health care community. It is estimated that medication error is responsible for 7,000 US deaths annually (1999 Institute of Medicine report, “To Err is Human: Building a Safer Health System,”).

In the practice of anesthesiology, physicians and nurse practitioners prepare syringes of drugs, which are used throughout the induction, and maintenance of general anesthesia, sedation and conscious sedation. The syringes are prepared by withdrawing the drugs from clear glass vials into plastic disposable syringes. In current practice, the practitioner often places a preprinted label or handmade mark on the syringe that details the contents. The labels are preprinted with the name of the drug and are color coded by drug class. However, color coded syringe labels identify otherwise indistinguishable syringes of liquid only when applied correctly by the end-user. When syringe is mislabeled, inappropriate drug use could be catastrophic for the patient. Most drug solutions have the appearance and consistency of water once drawn into a syringe. The opportunity for error arises when the physician or nurse practitioner places a preprinted label, handmade mark, or attempts to memorize which drug solution is in a given syringe. Over the course of a busy day, an overnight shift, or emergency situation, the potential for mislabeled syringes rises sharply. The central deficiency of the current system is that it is impossible to determine the contents of a syringe aside from looking at the label placed on the syringe by an individual health care provider.

Based on the foregoing, there is a need for safer solutions of injectable drugs and methods of use.

Atracurium besylate is the generic name for the compound 2,2′-(3,11-dioxo-4,10-dioxamidecamethylene) bis(1,2,3,4-tetrahydro-6,7-dimethoxy-2-methyl-1-veratrylisoquinolinium) di(benzenesulfonate), a useful injectable muscle relaxant. It is also known by the tradename TRACRIUM®. The chemical structure of atracurium besylate is shown in Formula 1.

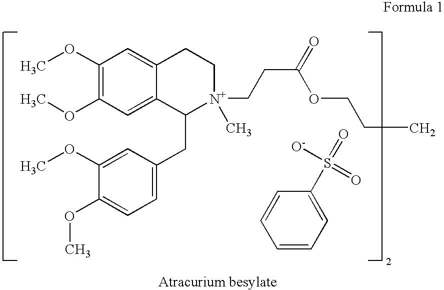

Atracurium besylate is used as an adjunct to general anesthesia, to facilitate endotracheal intubation and to provide skeletal muscle relaxation during surgery or mechanical ventilation.

Rocuronium is the generic name for the compound 1-[17β-(acetyloxy)-3α-hydroxy-2β-(4-morpholinyl)-5a-androstan-16β-yl]-1-(2-propenyl)pyrrolidinium, a useful injectable muscle relaxant. Rocuronium is described in U.S. Pat. No. 4,894,369, which is incorporated herein, by reference in its entireity. It is also known by the tradenames ZEMURON® and ESMERON®. The chemical structure of rocuronium bromide is shown in Formula 2.

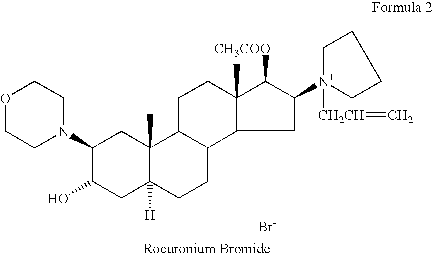

Rocuronium is used as an adjunct to general anesthesia, to facilitate mechanical ventilation, and to provide skeletal muscle relaxation during surgery or maniplulative procedures.

Vecuronium is the generic name for the compound (+)-1-(3,17-diacetoxy-2-piperidino-5-androstan-16-yl)-1-methylpiperidinium bromide, a useful injectable muscle relaxant. It is also known by the tradenames NORCURON®, and VECURON®. The chemical structure of vecuronium bromide is shown in Formula 3.

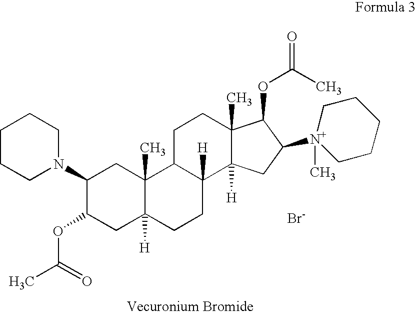

Vecuronium is used as an adjunct to general anesthesia, to facilitate mechanical ventilation, and to provide skeletal muscle relaxation during surgery or maniplulative procedures.

Cisatracurium besylate is the generic name for the compound [1R-[1α,2α(1′R*,2′*)]]-2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methylisoquinolinium]di(benzenesulfonate), a useful injectable muscle relaxant. The chemical structure of cisatracurium besylate is shown in Formula 4.

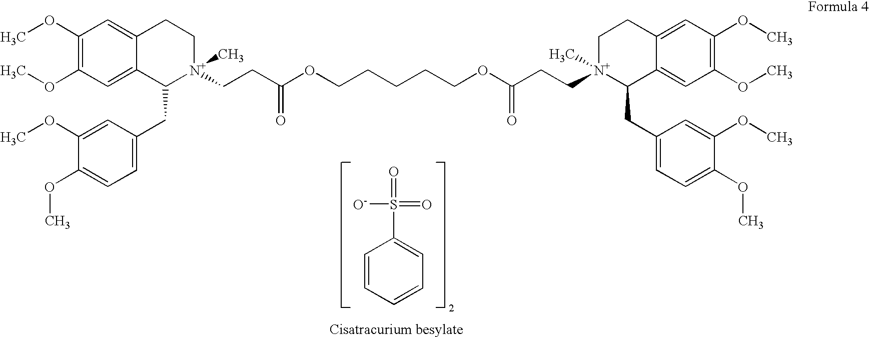

Cisatracurium besylate is used as an adjunct to general anesthesia, to facilitate endotracheal intubation and to provide skeletal muscle relaxation during surgery or mechanical ventilation.

Pancuronium is the generic name for the compound 3α,17β-diacetoxy-5α-androstan-2β,16β-ylene bis[1-methylpiperidinium], a useful injectable muscle relaxant. It is also known by the tradename PAVULON®. The chemical structure of pancuronium bromide is shown in Formula 5.

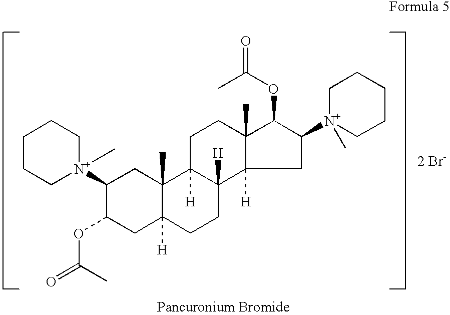

Pancuronium is used as an adjunct to general anesthesia, to facilitate mechanical ventilation, and to provide skeletal muscle relaxation during surgery or maniplulative procedures.

Succinylcholine is the generic name for the compound 2,2′-[(1,4-dioxo-1,4-butanediyl)bis(oxy)]bis[N,N,N-trimethylethanaminium], a useful injectable muscle relaxant. It is also known as suxamethonium chloride, scoline, or by the tradenames ANECTINE®, QUELICIN®, FLO-PACK®, and SUCOSTRIN®. The chemical structure of succinylcholine dichloride is shown in Formula 6.

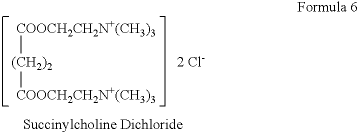

Succinylcholine is used as an adjunct to general anesthesia, to facilitate mechanical ventilation, and to provide skeletal muscle relaxation during surgery or manipulative procedures.

Lorazepam is the generic name for the compound 7-chloro-5-(o-chlorophenyl)-1,3-dihydro-3-hydroxy-2H-1,4-benzodiazepin-2-one. It is also known by the tradenames VERSED®, and ATIVAN®. The chemical structure of lorazepam is shown in Formula 7.

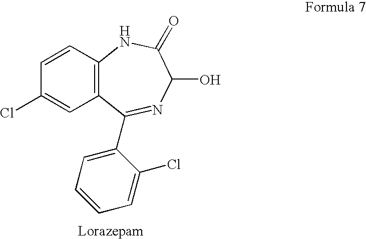

Injectable lorazepam is used for the following indications:
i. As premedication to relieve anxiety and tension, and to diminish recall of events associated with major or minor surgical and diagnostic procedures.
ii. Symptomatic relief of acute anxiety
iii. Treatment of status epilepticus caused by various partial and generalized types. Among the seizures known to respond to lorazepam injection are: generalized (tonic-clonic, “grand-mal”) seizures, generalized absence (“petit mal”) seizures or spike-wave stupor, partial elementary (focal motor) seizures, partial complex (psychomotor) seizures, and combinations such as generalized seizures with focal onset.
iv. Initial treatment with lorazepam injection results in prolonged cessation of seizure activity.

Midazolam is the generic name for the compound 8-chloro-6-(2-fluorophenyl)-1-methyl-4H-Imidazo(1,5-a)(1,4)benzodiazepine. It is also known by the tradenames VERSED®, SEDEVEN®, HYPNOVEL®, and DORMICUM®. The chemical structure of the free base form of midazolam is shown in Formula 8.

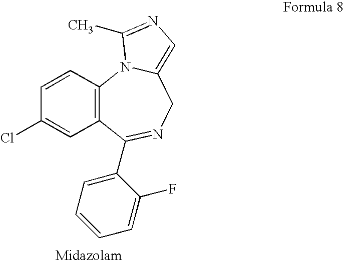

Intravenous midazolam hydrochloride is used for the induction of sedation before general anesthesia, induction of general anesthesia, and to impair the memory of perioperative patients (anterograde amnesia). Midazolam hydrochloride may also be used for conscious sedation prior to short diagnostic and endoscopic procedures, and as the hypnotic supplement to inhaled volatile agents, nitrous oxide and oxygen (balanced anesthesia) for short surgical procedures.

Diazepam is the generic name for the compound 7-chloro-1,3-dihydro-1-methyl-5-phenyl-2H-1,4-benzodiazepin-2-one. It is also known by the tradename VALIUM®. The chemical structure of diazepam is shown in Formula 9.

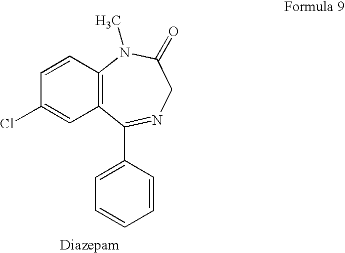

Diazepam is used for the following indications:
i. As premedication and/or cardioversion to relieve anxiety and tension, and to diminish recall of events associated with major or minor surgical and diagnostic procedures.
ii. Symptomatic relief of acute anxiety
iii. Treatment of status epilepticus caused by various partial and generalised types. Among the seizures known to respond to diazepam injection are: generalised (tonic-clonic, “grand-mal”) seizures, generalised absence (“petit mal”) seizures or spike-wave stupor, partial elementary (focal motor) seizures, partial complex (psychomotor) seizures, and combinations such as generalised seizures with focal onset.
iv. Initial treatment with diazepam injection results in prolonged cessation of seizure activity.
v. Symptomatic relief of acute agitation, tremor, impending or acute delirium tremens, and hallucinations due to acute alcohol withdrawal
vi. Treatment of muscle spasms associated with local pathology, cerebral palsy, athetosis, stiff-man syndrome, or tetanus.

Etomidate is the generic name for the compound (R)-(+)-ethyl-1-(1-phenylethyl)-1H-imidazole-5-carboxylate, a useful injectable anesthesia induction agent. It is also known by the tradenames AMIDATE®, and HYPNOMIDATE®. The chemical structure of etomidate is shown in Formula 10.

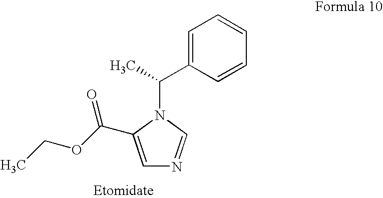

Etomidate is used for the induction of general anesthesia.

Propofol is the generic name for the compound 2,6-diisopropylphenol, a useful injectable anesthesia induction agent. It is also known by the tradenames DIPRLVAN®, and DIPRIVAN VHA PLUS®. The chemical structure of propofol is shown in Formula 11.

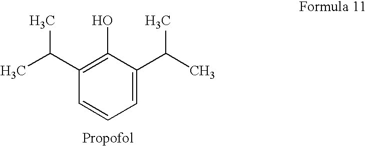

Propofol is used for the induction and maintenance of general anesthesia, and the induction and maintenance of sedation. Solutions of propofol are described in U.S. Pat. No. 6,326,406, 20020107291, A1, 20040014718 A1, 20050004234 A1, and 20040235964 A1, which are incorporated herein by reference.

Fospropofol disodium is the generic name for the compound disodium (2,6-diisopropylphenoxy)methyl phosphate, a prodrug of the useful injectable anesthesia induction agent propofol. It is also known by the tradename AQUAVAN®. The chemical structure of fospropofol disodium is shown in Formula 12.

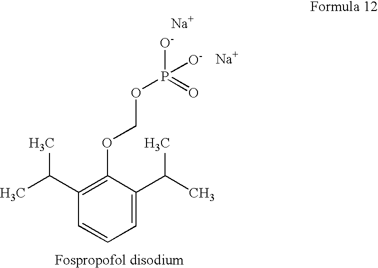

Fospropofol disodium is used for the induction and maintenance of general anesthesia, and the induction and maintenance of sedation. Solutions of fospropofol disodium are described in U.S. Pat. No. 6,204,257, which is incorporated herein by reference.

Ketamine is the generic name for the compound 2-(2-chlorophenyl)-2-(methylamino)-cyclohexanone, a useful injectable anesthesia induction agent. It is also known by the tradenames KETANEST®, KETASET®, and KETALAR®. The chemical structure of the free base from of ketamine is shown in Formula 13.

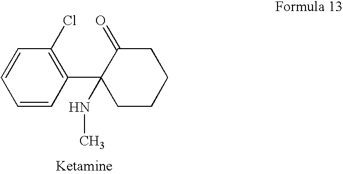

Ketamine hydrochloride is used for the induction and maintenance of general anesthesia.

Atropine is a tropane alkaloid extracted from the deadly nightshade (Atropa belladonna) and other plants of the family Solanaceae. It is also known as hyoscyamine, and by the tradename ATROPEN®. The chemical structure of atropine is shown in Formula 14.

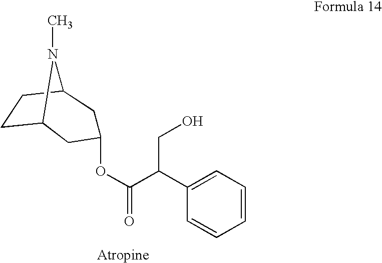

Injectable atropine, most commonly atropine sulfate monohydrate, is used as a preanesthetic to reduce salivary, and bronchial secretions, to treat reflex bradycardia, and in the emergent treatment of cardiac dysrhythmias.

Glycopyrrolate is the generic name for the compound 3-[(cyclopentylhydroxyphenylacetyl)oxy]-1,1-dimethylpyrrolidinium bromide, a useful injectable anticholinergic. It is also known by the tradename ROBINUL®. The chemical structure of glycopyrrolate is shown in Formula 15.

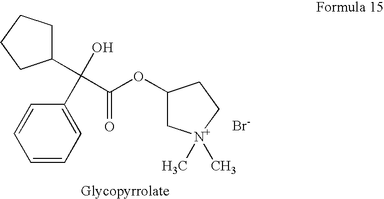

Injectable glycopyrrolate is used as a preoperative antimuscarinic to reduce salivary, tracheobronchial, and pharyngeal secretions; to reduce the volume and free acidity of gastric secretions; and to block cardiac vagal inhibitory reflexes during the induction of anesthesia and intubation.

### SUMMARY OF THE INVENTION

A colored drug solution will assist the practitioner avoid the confusion of one drug class with another. Such coloring agents are added to the solution for the purpose of reducing error on the part of heath care providers.

The invention is directed to pharmaceutical compositions comprising colored solutions, colored emulsions, or colored powders of injectable pharmaceuticals wherein said pharmaceuticals are selected from the group consisting of muscle relaxants, hypnotics, induction agents, and anticholinergics. The formulations of the present invention may all be colored using fluorescein. Different colors may be achieved by either varying the concentration of fluorescein, or by combining fluorescein with another dye. The invention is also directed to methods involving the use of said pharmaceutical compositions.

An additional benefit of colored drug solutions is that they allow a practitioner to visually confirm that injected drug has cleared the tubing and entered a patient's vein.

### DETAILED DESCRIPTION OF THE INVENTION

The invention is directed to a colored injectable pharmaceutical, wherein said color is derived from a dye comprising fluorescein.

An embodiment of the invention is a colored injectable pharmaceutical, wherein said color is derived from a dye comprising fluorescein and methylene blue.

An embodiment of the invention is a colored injectable pharmaceutical, wherein said color is derived from a dye comprising fluorescein and indigo carmine.

An embodiment of the invention is a colored injectable pharmaceutical, wherein said color is derived from a dye comprising fluorescein, and wherein the color is selected from the group consisting of yellow, orange, bright orange, and green.

An embodiment of the invention is a colored injectable pharmaceutical, wherein said color is derived from a dye comprising fluorescein and methylene blue, and wherein the color is green.

An embodiment of the invention is a colored injectable pharmaceutical, wherein said color is derived from a dye comprising fluorescein and indigo carmine, and wherein the color is green.

An embodiment of the invention is a colored injectable muscle relaxant, wherein said color is derived from a dye comprising fluorescein in the concentration of 7 mg/mL to 250 mg/mL, and wherein the color is bright orange.

An embodiment of the invention is a colored injectable muscle relaxant, wherein said color is derived from a dye comprising fluorescein in the concentration of 7 mg/mL to 100 mg/mL, and wherein the color is bright orange.

An embodiment of the invention is a colored injectable hypnotic, wherein said color is derived from a dye comprising fluorescein in the concentration of 1 mg/mL to 10 mg/mL, and wherein the color is orange.

An embodiment of the invention is a colored injectable hypnotic, wherein said color is derived from a dye comprising fluorescein in the concentration of 1.5 mg/mL to 2.5 mg/mL, and wherein the color is orange.

An embodiment of the invention is a colored injectable induction agent, wherein said color is derived from a dye comprising fluorescein in the concentration of 0.001 mg/mL to 2.5 mg/mL, and wherein the color is yellow.

An embodiment of the invention is a colored injectable induction agent, wherein said color is derived from a dye comprising fluorescein in the concentration of 0.05 mg/mL to 1.0 mg/mL, and wherein the color is yellow.

An embodiment of the invention is a colored injectable anticholinergic, wherein said color is derived from a dye comprising fluorescein in the concentration of 0.001 mg/mL to 0.1 mg/mL and methylene blue in the concentration of 0.001 mg/mL to 0.05 mg/mL, and wherein the color is green.

An embodiment of the invention is a colored injectable anticholinergic, wherein said color is derived from a dye comprising fluorescein in the concentration of 0.002 mg/mL to 0.02 mg/mL and methylene blue in the concentration of 0.002 mg/mL to 0.02 mg/mL, and wherein the color is green.

An embodiment of the invention is a colored injectable anticholinergic, wherein said color is derived from a dye comprising fluorescein in the concentration of 0.001 mg/mL to 0.4 mg/mL and indigo carmine in the concentration of 0.001 mg/mL to 0.05 mg/mL, and wherein the color is green.

An embodiment of the invention is a colored injectable anticholinergic, wherein said color is derived from a dye comprising fluorescein in the concentration of 0.002 mg/mL to 0.2 mg/mL and indigo carmine in the concentration of 0.001 mg/mL to 0.03 mg/mL, and wherein the color is green.

An embodiment of the invention is a method of preparing color coded injectable pharmaceuticals, wherein different colors are created by varying the concentration of fluorescein.

An embodiment of the invention is a method of preparing color coded injectable pharmaceuticals, wherein the colors yellow, orange, and bright orange are created by varying the concentration of fluorescein.

An embodiment of the invention is a pharmaceutical solution comprising a colored solution of atracurium besylate.

An embodiment of the invention is a pharmaceutical solution which comprises a bright orange solution of atracurium besylate.

An embodiment of the invention is a pharmaceutical solution which comprises a bright orange solution of atracurium besylate, wherein said orange color is derived from fluorescein.

An embodiment of the invention is a pharmaceutical solution which comprises a bright orange solution aqueous of atracurium besylate, wherein said orange color is derived from fluorescein.

In one embodiment, the present invention provides a pharmaceutical solution which includes a therapeutically effective amount of a colored solution of atracurium besylate for eliciting a muscle relaxant response in a mammal.

In another embodiment, the present invention provides a method for eliciting a muscle relaxant response in a mammal which includes administering a therapeutically effective amount of a colored solution of atracurium besylate to the mammal that is sufficient to elicit a muscle relaxant response.

In another embodiment, the present invention provides a method for eliciting a muscle relaxant response in a mammal which includes administering a therapeutically effective amount of a bright orange aqueous solution of atracurium besylate, wherein the bright orange color of said solution is derived from fluorescein.

In a particularly preferred embodiment, the present invention provides a pharmaceutical solution comprising water, atracurium besylate in the range of 0.1 mg/mL to 250 mg/mL and fluorescein in the range of 7 to 250 mg/mL.

In a particularly preferred embodiment, the present invention provides a pharmaceutical solution comprising water, atracurium besylate in the range of 1 mg/mL to 50 mg/mL, and fluorescein in the range of 7 to 100 mg/mL.

In a particularly preferred embodiment, the present invention provides a pharmaceutical solution comprising water, atracurium besylate in the concentration of 10 mg/mL, and fluorescein in the concentration of 10 mg/mL.

An embodiment of the invention is a pharmaceutical composition comprising a colored solution of rocuronium.

An embodiment of the invention is a pharmaceutical composition which comprises a bright orange solution of rocuronium.

An embodiment of the invention is a pharmaceutical composition which comprises a bright orange solution of rocuronium, wherein said bright orange color is derived from fluorescein.

An embodiment of the invention is a pharmaceutical composition which comprises an aqueous bright orange solution of rocuronium, wherein said bright orange color is derived from fluorescein.

In one embodiment, the present invention provides a pharmaceutical composition which includes a therapeutically effective amount of a colored solution of rocuronium bromide for eliciting a muscle relaxant response in a mammal.

In another embodiment, the present invention provides a method for eliciting a muscle relaxant response in a mammal which includes administering a therapeutically effective amount of a colored solution of rocuronium bromide to the mammal that is sufficient to elicit a muscle relaxant response.

In another embodiment, the present invention provides a method for eliciting a muscle relaxant response in a mammal which includes administering a therapeutically effective amount of a bright orange aqueous solution of rocuronium bromide, wherein said bright orange color is derived from fluorescein.

In a particularly preferred embodiment, the present invention provides a pharmaceutical solution comprising water, rocuronium bromide in the range of 0.01 mg/mL to 100 mg/mL and fluorescein in the range of 7 to 250 mg/mL.

In a particularly preferred embodiment, the present invention provides a pharmaceutical solution comprising water, rocuronium bromide in the range of 0.5 mg/mL to 10 mg/mL, and fluorescein in the range of 5 to 25 mg/mL.

In a particularly preferred embodiment, the present invention provides a pharmaceutical solution comprising water, rocuronium bromide in the range of 0.5 mg/mL to 10 mg/mL, and fluorescein in the range of 7 to 100 mg/mL.

In a particularly preferred embodiment, the present invention provides a pharmaceutical solution comprising water, rocuronium bromide in the concentration of 1 mg/mL, and fluorescein in the concentration of 10 mg/mL.

An embodiment of the invention is a pharmaceutical composition comprising a colored vecuronium powder.

An embodiment of the invention is a colored vecuronium powder, wherein said color is bright orange.

An embodiment of the invention is a bright orange vecuronium powder, wherein said bright orange color is derived from fluorescein.

An embodiment of the invention is a bright orange vecuronium powder, wherein said bright orange color is derived from fluorescein for use as a medicament.

An embodiment of the invention is a method for eliciting a muscle relaxant response in a mammal comprising administering a therapeutically effective amount of a bright orange solution of vecuronium bromide to the mammal that is sufficient to elicit a muscle relaxant response, wherein said bright orange color is derived from fluorescein.

An embodiment of the invention is a pharmaceutical solution comprising a colored solution of cisatracurium besylate.

An embodiment of the invention is a pharmaceutical solution which comprises a bright orange solution of cisatracurium besylate.

An embodiment of the invention is a pharmaceutical solution which comprises a bright orange solution of cisatracurium besylate, wherein said orange color is derived from fluorescein.

An embodiment of the invention is a pharmaceutical solution which comprises a bright orange solution aqueous of cisatracurium besylate, wherein said orange color is derived from fluorescein.

In one embodiment, the present invention provides a pharmaceutical solution which includes a therapeutically effective amount of a colored solution of cisatracurium besylate for eliciting a muscle relaxant response in a mammal.

In another embodiment, the present invention provides a method for eliciting a muscle relaxant response in a mammal which includes administering a therapeutically effective amount of a colored solution of cisatracurium besylate to the mammal that is sufficient to elicit a muscle relaxant response.

In another embodiment, the present invention provides a method for eliciting a muscle relaxant response in a mammal which includes administering a therapeutically effective amount of a bright orange aqueous solution of cisatracurium besylate, wherein the bright orange color of said solution is derived from fluorescein.

In a particularly preferred embodiment, the present invention provides a pharmaceutical solution comprising water, cisatracurium besylate in the range of 0.1 mg/mL to 250 mg/mL and fluorescein in the range of 7 to 250 mg/mL.

In a particularly preferred embodiment, the present invention provides a pharmaceutical solution comprising water, cisatracurium besylate in the range of 0.5 mg/mL to 25 mg/mL, and fluorescein in the range of 7 to 100 mg/mL.

In a particularly preferred embodiment, the present invention provides a pharmaceutical solution comprising water, cisatracurium besylate in the concentration of 2 mg/mL, and fluorescein in the concentration of 10 mg/mL.

An embodiment of the invention is a pharmaceutical solution comprising a colored solution of pancuronium.

An embodiment of the invention is a pharmaceutical solution which comprises a bright orange solution of pancuronium.

In a preferred embodiment, the present invention provides a pharmaceutical solution which comprises a bright orange solution of pancuronium, wherein said bright orange color is derived from fluorescein.

In a preferred embodiment, the present invention provides a pharmaceutical solution which comprises a bright orange aqueous solution of pancuronium, wherein said bright orange color is derived from fluorescein.

In one embodiment, the present invention provides a pharmaceutical solution which includes a therapeutically effective amount of a colored solution of pancuronium bromide for eliciting a muscle relaxant response in a mammal.

In another embodiment, the present invention provides a method for eliciting a muscle relaxant response in a mammal which includes administering a therapeutically effective amount of a colored solution of pancuronium bromide to the mammal that is sufficient to elicit a muscle relaxant response.

In another embodiment, the present invention provides a method for eliciting a muscle relaxant response in a mammal which includes administering a therapeutically effective amount of a bright orange aqueous solution of pancuronium bromide, wherein the bright orange color of said solution is derived from fluorescein.

In a particularly preferred embodiment, the present invention provides a pharmaceutical solution comprising water, pancuronium bromide in the range of 0.01 mg/mL to 100 mg/mL and fluorescein in the range of 7 to 250 mg/mL.

In a particularly preferred embodiment, the present invention provides a pharmaceutical solution comprising water, pancuronium bromide in the range of 0.5 mg/mL to 10 mg/mL, and fluorescein in the range of 5 to 25 mg/mL.

In a particularly preferred embodiment, the present invention provides a pharmaceutical solution comprising water, pancuronium bromide in the range of 0.5 mg/mL to 10 mg/mL, and fluorescein in the range of 7 to 100 mg/mL.

In a particularly preferred embodiment, the present invention provides a pharmaceutical solution comprising water, pancuronium bromide in the concentration of 1 mg/mL, and fluorescein in the concentration of 10 mg/mL.

An embodiment of the invention is a pharmaceutical solution comprising a colored solution of succinylcholine.

An embodiment of the invention is a pharmaceutical solution which comprises a bright orange solution of succinylcholine.

An embodiment of the invention is a pharmaceutical solution which comprises a bright orange solution of succinylcholine, wherein said orange color is derived from fluorescein.

An embodiment of the invention is a pharmaceutical solution which comprises a bright orange solution aqueous of succinylcholine, wherein said orange color is derived from fluorescein.

In one embodiment, the present invention provides a pharmaceutical composition which includes a therapeutically effective amount of a colored solution of succinylcholine dichloride for eliciting a muscle relaxant response in a mammal.

In another embodiment, the present invention provides a method for eliciting a muscle relaxant response in a mammal which includes administering a therapeutically effective amount of a colored solution of succinylcholine dichloride to the mammal that is sufficient to elicit a muscle relaxant response.

In another embodiment, the present invention provides a method for eliciting a muscle relaxant response in a mammal which includes administering a therapeutically effective amount of a bright orange aqueous solution of succinylcholine dichloride, wherein the bright orange color of said solution is derived from fluorescein.

In a particularly preferred embodiment, the present invention provides a pharmaceutical solution comprising water, succinylcholine dichloride in the range of 1 mg/mL to 250 mg/mL and fluorescein in the range of 7 to 250 mg/mL.

In a particularly preferred embodiment, the present invention provides a pharmaceutical solution comprising water, succinylcholine dichloride in the range of 20 mg/mL to 100 mg/mL, and fluorescein in the range of 7 to 100 mg/mL.

In a particularly preferred embodiment, the present invention provides a pharmaceutical solution comprising water, succinylcholine dichloride in the concentration of 20 mg/mL, and fluorescein in the concentration of 10 mg/mL.

An embodiment of the invention is a pharmaceutical composition comprising a colored solution of lorazepam.

An embodiment of the invention is a pharmaceutical composition which comprises an orange solution of lorazepam.

An embodiment of the invention is a pharmaceutical composition which comprises an orange solution of lorazepam, wherein said orange color is derived from fluorescein.

In a preferred embodiment, the present invention provides a pharmaceutical composition which comprises an orange solution of lorazepam in propylene glycol and polyethylene glycol, wherein the orange color is derived from fluorescein.

An embodiment of the invention is a pharmaceutical composition which comprises a propylene glycol and polyethylene glycol solution of lorazepam, wherein the orange color is derived from fluorescein, for use as a medicament.

In a particularly preferred embodiment, the present invention provides a pharmaceutical solution comprising a propylene glycol, polyethylene glycol, and benzyl alcohol solution of lorazepam, in the range of 0.5 mg/mL to 50 mg/mL and fluorescein in the range of 1.0 to 10 mg/mL.

In a particularly preferred embodiment, the present invention provides a pharmaceutical solution comprising a propylene glycol, polyethylene glycol, and benzyl alcohol solution of lorazepam, in the range of 1 mg/mL to 10 mg/mL, and fluorescein in the range of 1.5 to 5 mg/mL.

In a particularly preferred embodiment, the present invention provides a pharmaceutical solution comprising propylene glycol, polyethylene glycol, and benzyl alcohol solution of lorazepam, in the concentration of about 2 mg/mL, and fluorescein in the concentration of about 2.5 mg/mL.

In another embodiment, the present invention provides a method for inducing anterograde amnesia in a mammal which includes administering a colored solution of lorazepam to the mammal that is sufficient to induce anterograde amnesia.

In another embodiment, the present invention provides a method for treatment of epilepsy in a mammal which includes administering a colored solution of lorazepam to the mammal that is sufficient to control seizures.

In another embodiment, the present invention provides a method for treatment of anxiety in a mammal which includes administering a colored solution of lorazepam to the mammal that is sufficient to provide relief from anxiety.

In another embodiment, the present invention provides a method for inducing anterograde amnesia in a mammal which includes administering an orange solution of lorazepam, wherein the orange color is derived from fluorescein.

In another embodiment, the present invention provides a method for treatment of epilepsy in a mammal which includes administering an orange solution of lorazepam, wherein the orange color is derived from fluorescein.

In another embodiment, the present invention provides a method for treatment of anxiety in a mammal which includes administering an orange solution of lorazepam, wherein the orange color is derived from fluorescein.

An embodiment of the invention is a pharmaceutical composition comprising a colored solution of midazolam hydrochloride.

An embodiment of the invention is a pharmaceutical composition which comprises an orange solution of midazolam hydrochloride.

In a preferred embodiment, the present invention provides a pharmaceutical composition which comprises an aqueous solution of midazolam hydrochloride, wherein the orange color is derived from fluorescein.

An embodiment of the invention is a pharmaceutical composition which comprises an aqueous solution of midazolam hydrochloride, wherein the orange color is derived from fluorescein.

An embodiment of the invention is a pharmaceutical composition which comprises an aqueous solution of midazolam hydrochloride, wherein the orange color is derived from fluorescein, for use as a medicament.

In a particularly preferred embodiment, the present invention provides a pharmaceutical solution comprising water, midazolam hydrochloride in the range of 0.1 mg/mL to 50 mg/mL and fluorescein in the range of 1 to 10 mg/mL.

In a particularly preferred embodiment, the present invention provides a pharmaceutical solution comprising water, midazolam hydrochloride in the range of 0.5 mg/mL to 10 mg/mL, and fluorescein in the range of 1.5 to 5 mg/mL.

In a particularly preferred embodiment, the present invention provides a pharmaceutical solution comprising water, midazolam hydrochloride in the concentration of 1 mg/mL, and fluorescein in the concentration of 2.5 mg/mL.

In another embodiment, the invention comprises a pharmaceutical solution comprising water, midazolam hydrochloride in the range of 0.5 mg/mL to 10 mg/mL, fluorescein in the range of 1.5 to 5 mg/mL, further comprising sodium chloride, wherein the sodium chloride concentration is less than 10% (w/v).

In another embodiment, the invention comprises a pharmaceutical solution comprising water, midazolam hydrochloride in the range of 0.5 mg/mL to 10 mg/mL, fluorescein in the range of 1.5 to 5 mg/mL, less than 10% (w/v) sodium chloride, further comprising disodium edentate wherein the disodium edentate concentration is less than (1% w/v).

In another embodiment, the invention comprises a pharmaceutical solution comprising water, midazolam hydrochloride in the range of 0.5 mg/mL to 10 mg/mL, fluorescein in the range of 1.5 to 5 mg/mL, less than 10% (w/v) sodium chloride, less than (1% w/v) disodium edentate, further comprising hydrochloric acid to adjust the pH to about 3.

In another embodiment, the invention comprises a pharmaceutical solution comprising water, midazolam hydrochloride in the range of 0.5 mg/mL to 10 mg/mL, fluorescein in the range of 1.5 to 5 mg/mL, less than 10% (w/v) sodium chloride, less than (1% w/v) disodium edentate, hydrochloric acid to adjust the pH to about 3, further comprising less than (5% w/v) benzyl alcohol.

In another embodiment, the present invention provides a method for inducing sedation in a mammal which includes administering a colored solution of midazolam hydrochloride to the mammal that is sufficient to induce sedation.

In another embodiment, the present invention provides a method for inducing anesthesia in a mammal which includes administering a colored solution of midazolam hydrochloride to the mammal that is sufficient to induce anesthesia.

In another embodiment, the present invention provides a method for inducing anterograde amnesia in a mammal which includes administering a colored solution of midazolam hydrochloride to the mammal that is sufficient to induce anterograde amnesia.

In another embodiment, the present invention provides a method for inducing sedation in a mammal which includes administering an orange aqueous solution of midazolam hydrochloride, wherein said orange color is derived from fluorescein.

In another embodiment, the present invention provides a method for inducing anesthesia in a mammal which includes administering an orange aqueous solution of midazolam hydrochloride, wherein said orange color is derived from fluorescein.

In another embodiment, the present invention provides a method for inducing anterograde amnesia in a mammal which includes administering an orange aqueous solution of midazolam hydrochloride, wherein said orange color is derived from fluorescein.

An embodiment of the invention is a pharmaceutical composition comprising a colored injectable formulation of diazepam.

An embodiment of the invention is a pharmaceutical composition comprising an orange solution of diazepam.

An embodiment of the invention is a pharmaceutical composition comprising an orange emulsion of diazepam.

An embodiment of the invention is a pharmaceutical composition which comprises an orange solution of diazepam, wherein said orange color is derived from fluorescein.

An embodiment of the invention is a pharmaceutical composition which comprises an orange emulsion of diazepam, wherein said orange color is derived from fluorescein.

In a preferred embodiment, said pharmaceutical composition comprises an orange propylene glycol and polyethylene glycol solution of diazepam, wherein the orange color is derived from fluorescein.

In a preferred embodiment, said pharmaceutical composition comprises an orange propylene glycol and ethyl alcohol solution of diazepam, wherein the orange color is derived from fluorescein.

In a preferred embodiment, said pharmaceutical composition comprises an orange soybean oil and water emulsion of diazepam, wherein the orange color is derived from fluorescein.

An embodiment of the invention is a pharmaceutical composition which comprises an orange propylene glycol and polyethylene glycol solution of diazepam, wherein the orange color is derived from fluorescein, for use as a medicament.

An embodiment of the invention is a pharmaceutical composition which comprises an orange propylene glycol and polyethylene glycol emulsion of diazepam, wherein the orange color is derived from fluorescein, for use as a medicament.

In a particularly preferred embodiment, the present invention provides a solution comprising propylene glycol, polyethylene glycol, diazepam in the range of 0.5 mg/mL to 50 mg/mL and fluorescein in the range of 1.0 to 10 mg/mL.

In a particularly preferred embodiment, the present invention provides a solution comprising propylene glycol, polyethylene glycol, diazepam in the range of 1 mg/mL to 10 mg/mL, and fluorescein in the range of 1.5 to 5 mg/mL.

In a particularly preferred embodiment, the present invention provides a solution comprising propylene glycol, polyethylene glycol, diazepam in the concentration of about 4 mg/mL, and fluorescein in the concentration of about 2.5 mg/mL.

In a particularly preferred embodiment, the present invention provides a solution comprising propylene glycol, ethyl alcohol, diazepam in the range of 0.5 mg/mL to 50 mg/mL and fluorescein in the range of 1.0 to 10 mg/mL.

In a particularly preferred embodiment, the present invention provides a solution comprising propylene glycol, ethyl alcohol, diazepam in the range of 1 mg/mL to 10 mg/mL, and fluorescein in the range of 1.5 to 5 mg/mL.

In a particularly preferred embodiment, the present invention provides a solution comprising propylene glycol, ethyl alcohol, diazepam in the concentration of about 5 mg/mL, and fluorescein in the concentration of about 2.5 mg/mL.

In a particularly preferred embodiment, the present invention provides an emulsion comprising soybean oil, water, diazepam in the range of 0.5 mg/mL to 50 mg/mL and fluorescein in the range of 1.0 to 10 mg/mL.

In a particularly preferred embodiment, the present invention provides an emulsion comprising soybean oil, water, diazepam in the range of 1 mg/mL to 10 mg/mL, and fluorescein in the range of 1.5 to 5 mg/mL.

In a particularly preferred embodiment, the present invention provides an emulsion comprising soybean oil, water, diazepam in the concentration of about 5 mg/mL, and fluorescein in the concentration of about 2.5 mg/mL.

In another embodiment, the present invention provides a method for inducing anterograde amnesia in a mammal which includes administering a colored solution of diazepam to the mammal that is sufficient to induce anterograde amnesia.

In another embodiment, the present invention provides a method for preoperatively relieving anxiety and tension in a mammal which includes administering a colored solution of diazepam to the mammal that is sufficient to preoperatively relieve anxiety and tension.

In another embodiment, the present invention provides a method for relieving acute anxiety and tension in a mammal which includes administering a colored solution of diazepam to the mammal that is sufficient to relieve acute anxiety.

In another embodiment, the present invention provides a method for relieving status epilepticus in a mammal which includes administering a colored solution of diazepam to the mammal that is sufficient to relieve status epilepticus.

In another embodiment, the present invention provides a method for relieving acute agitation, tremor, impending or acute delirium tremens, and hallucinations due to acute alcohol withdrawal in a mammal which includes administering a colored solution of diazepam to the mammal that is sufficient to relieve said symptoms of alcohol withdrawal.

In another embodiment, the present invention provides a method for relieving muscle spasms associated with local pathology, cerebral palsy, athetosis, stiff-man syndrome, or tetanus in a mammal which includes administering a colored solution of diazepam to the mammal that is sufficient to relieve said muscle spasms.

In another embodiment, the present invention provides a method for inducing anterograde amnesia in a mammal which includes administering an orange solution of diazepam, wherein said orange color is derived from fluorescein.

In another embodiment, the present invention provides a method for preoperatively relieving anxiety and tension in a mammal which includes administering an orange solution of diazepam, wherein said orange color is derived from fluorescein.

In another embodiment, the present invention provides a method for relieving acute anxiety and tension in a mammal which includes administering an orange solution of diazepam, wherein said orange color is derived from fluorescein.

In another embodiment, the present invention provides a method for relieving status epilepticus in a mammal which includes administering an orange solution of diazepam, wherein said orange color is derived from fluorescein.

In another embodiment, the present invention provides a method for relieving acute agitation, tremor, impending or acute delirium tremens, and hallucinations due to acute alcohol withdrawal in a mammal which includes administering an orange solution of diazepam, wherein said orange color is derived from fluorescein.

In another embodiment, the present invention provides a method for relieving muscle spasms associated with local pathology, cerebral palsy, athetosis, stiff-man syndrome, or tetanus in a mammal which includes administering an orange solution of diazepam, wherein said orange color is derived from fluorescein.

An embodiment of the invention is a pharmaceutical composition comprising a colored solution of etomidate.

An embodiment of the invention is a pharmaceutical composition which comprises a colored solution of etomidate.

An embodiment of the invention is a pharmaceutical composition which comprises a yellow solution of etomidate.

In another embodiment, the present invention provides a method for the induction of general anesthesia in a mammal which includes administering a therapeutically effective amount of a colored solution of etomidate to the mammal that is sufficient to induce general anesthesia.

In another embodiment, the present invention provides a method for the induction of general anesthesia in a mammal which includes administering a therapeutically effective amount of a yellow solution of etomidate, wherein the yellow color of said solution is derived from fluorescein.

In another embodiment, the present invention provides a method for the maintenance of general anesthesia in a mammal which includes administering a therapeutically effective amount of a colored solution of etomidate to the mammal that is sufficient to maintain general anesthesia.

In another embodiment, the present invention provides a method for the maintenance of general anesthesia in a mammal which includes administering a therapeutically effective amount of a yellow solution of etomidate, wherein the yellow color of said solution is derived from fluorescein.

In a preferred embodiment, the present invention provides a pharmaceutical composition which comprises a solution of water, propylene glycol, etomidate and fluorescein.

In another embodiment, the present invention provides a pharmaceutical composition which comprises a solution of water, propylene glycol, etomidate and fluorescein, for use as a medicament.

In a particularly preferred embodiment, the present invention provides a pharmaceutical solution comprising water, propylene glycol, etomidate in the range of 0.02 mg/mL to 20 mg/mL and fluorescein in the range of 0.001 to 2.5 mg/mL.

In a particularly preferred embodiment, the present invention provides a pharmaceutical solution comprising water, propylene glycol, etomidate in the range of 0.5 mg/mL to 10 mg/mL, and fluorescein in the range of 0.05 to 1 mg/mL.

In a particularly preferred embodiment, the present invention provides a pharmaceutical solution comprising water, propylene glycol, etomidate in the concentration of 2 mg/mL, and fluorescein in the concentration of 0.15 mg/mL.

An embodiment of the invention is a pharmaceutical composition comprising a colored solution or emulsion of propofol.

An embodiment of the invention is a pharmaceutical composition which comprises a colored solution or emulsion of propofol.

An embodiment of the invention is a pharmaceutical composition which comprises a yellow solution or emulsion of propofol.

In one embodiment, the present invention provides a pharmaceutical composition which includes a therapeutically effective amount of a colored solution or emulsion of propofol for the induction of general anesthesia in a mammal.

In another embodiment, the present invention provides a method for the induction of general anesthesia in a mammal which includes administering a therapeutically effective amount of a colored solution or emulsion of propofol to the mammal that is sufficient to induce general anesthesia.

In another embodiment, the present invention provides a method for the induction of sedation in a mammal which includes administering a therapeutically effective amount of a colored solution or emulsion of propofol to the mammal that is sufficient to induce sedation.

In another embodiment, the present invention provides a method for the maintenance of general anesthesia in a mammal which includes administering a therapeutically effective amount of a colored solution or emulsion of propofol to the mammal that is sufficient to maintain general anesthesia.

In another embodiment, the present invention provides a method for the maintenance of sedation in a mammal which includes administering a therapeutically effective amount of a colored solution or emulsion of propofol to the mammal that is sufficient to maintain sedation.

In another embodiment, the present invention provides a method for the induction of general anesthesia in a mammal which includes administering a therapeutically effective amount of a yellow solution or emulsion of propofol, wherein the yellow color of said solution or emulsion is derived from fluorescein.

In another embodiment, the present invention provides a method for the induction of sedation in a mammal which includes administering a therapeutically effective amount of a yellow solution or emulsion of propofol, wherein the yellow color of said solution or emulsion is derived from fluorescein.

In another embodiment, the present invention provides a method for the maintenance of general anesthesia in a mammal which includes administering a therapeutically effective amount of a yellow solution or emulsion of propofol, wherein the yellow color of said solution or emulsion is derived from fluorescein.

In another embodiment, the present invention provides a method for the maintenance of sedation in a mammal which includes administering a therapeutically effective amount of a yellow solution or emulsion of propofol, wherein the yellow color of said solution or emulsion is derived from fluorescein.

In a preferred embodiment, the present invention provides a pharmaceutical composition which comprises a solution or emulsion of water, soybean oil, propofol and fluorescein.

In another embodiment, the present invention provides a pharmaceutical composition which comprises a solution or emulsion of water, soybean oil, propofol and fluorescein, for use as a medicament.

In a particularly preferred embodiment, the present invention provides a solution or emulsion comprising water, propofol in the range of 1 mg/mL to 50 mg/mL and fluorescein in the range of 0.001 to 2.5 mg/mL.

In a particularly preferred embodiment, the present invention provides a solution or emulsion comprising water, propofol in the range of 5 mg/mL to 15 mg/mL, and fluorescein in the range of 0.05 to 1 mg/mL.

In a particularly preferred embodiment, the present invention provides a solution or emulsion comprising water, propofol in the concentration of 10 mg/mL, and fluorescein in the concentration of 0.15 mg/mL.

An embodiment of the invention is a pharmaceutical composition comprising a colored solution of fospropofol disodium.

An embodiment of the invention is a pharmaceutical composition which comprises a yellow solution of fospropofol disodium.

In one embodiment, the present invention provides a pharmaceutical composition which includes a therapeutically effective amount of a colored solution of fospropofol disodium for the induction of general anesthesia in a mammal.

In another embodiment, the present invention provides a method for the induction of general anesthesia in a mammal which includes administering a therapeutically effective amount of a colored solution of fospropofol disodium to the mammal that is sufficient to induce general anesthesia.

In another embodiment, the present invention provides a method for the induction of sedation in a mammal which includes administering a therapeutically effective amount of a colored solution of fospropofol disodium to the mammal that is sufficient to induce sedation.

In another embodiment, the present invention provides a method for the maintenance of general anesthesia in a mammal which includes administering a therapeutically effective amount of a colored solution of fospropofol disodium to the mammal that is sufficient to maintain general anesthesia.

In another embodiment, the present invention provides a method for the maintenance of sedation in a mammal which includes administering a therapeutically effective amount of a colored solution of fospropofol disodium to the mammal that is sufficient to maintain sedation.

In another embodiment, the present invention provides a method for the induction of general anesthesia in a mammal which includes administering a therapeutically effective amount of a yellow solution of fospropofol disodium, wherein the yellow color of said solution is derived from fluorescein.

In another embodiment, the present invention provides a method for the induction of sedation in a mammal which includes administering a therapeutically effective amount of a yellow solution of fospropofol disodium, wherein the yellow color of said solution is derived from fluorescein.

In another embodiment, the present invention provides a method for the maintenance of general anesthesia in a mammal which includes administering a therapeutically effective amount of a yellow solution of fospropofol disodium, wherein the yellow color of said solution is derived from fluorescein.

In another embodiment, the present invention provides a method for the maintenance of sedation in a mammal which includes administering a therapeutically effective amount of a yellow solution of fospropofol disodium, wherein the yellow color of said solution is derived from fluorescein.

In a preferred embodiment, the present invention provides a pharmaceutical composition which comprises an aqueous solution of fospropofol disodium and fluorescein.

In another embodiment, the present invention provides a pharmaceutical composition which comprises an aqueous solution of fospropofol disodium and fluorescein, for use as a medicament.

In a particularly preferred embodiment, the present invention provides a solution comprising water, fospropofol disodium in the range of 1 mg/mL to 50 mg/mL and fluorescein in the range of 0.001 to 2.5 mg/mL.

In a particularly preferred embodiment, the present invention provides a solution comprising water, fospropofol disodium in the range of 5 mg/mL to 15 mg/mL, and fluorescein in the range of 0.05 to 1 mg/mL.

In a particularly preferred embodiment, the present invention provides a solution comprising water, fospropofol disodium in the concentration of 10 mg/mL, and fluorescein in the concentration of 0.15 mg/mL.

An embodiment of the invention is a pharmaceutical composition comprising a colored solution of fospropofol disodium.

An embodiment of the invention is a pharmaceutical composition which comprises an orange solution of fospropofol disodium.

In one embodiment, the present invention provides a pharmaceutical composition which includes a therapeutically effective amount of a colored solution of fospropofol disodium for the induction of general anesthesia in a mammal.

In another embodiment, the present invention provides a method for the induction of general anesthesia in a mammal which includes administering a therapeutically effective amount of a colored solution of fospropofol disodium to the mammal that is sufficient to induce general anesthesia.

In another embodiment, the present invention provides a method for the induction of sedation in a mammal which includes administering a therapeutically effective amount of a colored solution of fospropofol disodium to the mammal that is sufficient to induce sedation.

In another embodiment, the present invention provides a method for the maintenance of general anesthesia in a mammal which includes administering a therapeutically effective amount of a colored solution of fospropofol disodium to the mammal that is sufficient to maintain general anesthesia.

In another embodiment, the present invention provides a method for the maintenance of sedation in a mammal which includes administering a therapeutically effective amount of a colored solution of fospropofol disodium to the mammal that is sufficient to maintain sedation.

In another embodiment, the present invention provides a method for the induction of general anesthesia in a mammal which includes administering a therapeutically effective amount of an orange solution of fospropofol disodium, wherein the orange color of said solution is derived from fluorescein.

In another embodiment, the present invention provides a method for the induction of sedation in a mammal which includes administering a therapeutically effective amount of an orange solution of fospropofol disodium, wherein the orange color of said solution is derived from fluorescein.

In another embodiment, the present invention provides a method for the maintenance of general anesthesia in a mammal which includes administering a therapeutically effective amount of an orange solution of fospropofol disodium, wherein the orange color of said solution is derived from fluorescein.

In another embodiment, the present invention provides a method for the maintenance of sedation in a mammal which includes administering a therapeutically effective amount of an orange solution of fospropofol disodium, wherein the orange color of said solution is derived from fluorescein.

In a preferred embodiment, the present invention provides a pharmaceutical composition which comprises an aqueous solution of fospropofol disodium and fluorescein.

In another embodiment, the present invention provides a pharmaceutical composition which comprises an aqueous solution of fospropofol disodium and fluorescein, for use as a medicament.

In a particularly preferred embodiment, the present invention provides a solution comprising water, fospropofol disodium in the range of 1 mg/mL to 50 mg/mL and fluorescein in the range of 1 to 10 mg/mL.

In a particularly preferred embodiment, the present invention provides a solution comprising water, fospropofol disodium in the range of 5 mg/mL to 15 mg/mL, and fluorescein in the range of 1.5 to 5 mg/mL.

In a particularly preferred embodiment, the present invention provides a solution comprising water, fospropofol disodium in the concentration of 10 mg/mL, and fluorescein in the concentration of 2.5 mg/mL.

The invention is directed to a pharmaceutical composition comprising a colored solution of ketamine.

An embodiment of the invention is a pharmaceutical composition which comprises a colored solution of ketamine hydrochloride.

An embodiment of the invention is a pharmaceutical composition which comprises a yellow solution of ketamine hydrochloride.

In one embodiment, the present invention provides a pharmaceutical composition which includes a therapeutically effective amount of a colored solution of ketamine hydrochloride for the induction of general anesthesia in a mammal.

In another embodiment, the present invention provides a method for the induction of general anesthesia in a mammal which includes administering a therapeutically effective amount of a colored solution of ketamine hydrochloride to the mammal that is sufficient to induce general anesthesia.

In another embodiment, the present invention provides a method for the induction of general anesthesia in a mammal which includes administering a therapeutically effective amount of a yellow aqueous solution of ketamine hydrochloride, wherein the yellow color of said solution is derived from fluorescein.

In a preferred embodiment, the present invention provides a pharmaceutical composition which comprises a solution of water, ketamine hydrochloride and fluorescein.

In another embodiment, the present invention provides a pharmaceutical composition which comprises a solution of water, ketamine hydrochloride and fluorescein, for use as a medicament.

In a particularly preferred embodiment, the present invention provides a pharmaceutical solution comprising water, ketamine hydrochloride in the range of 10 mg/mL to 500 mg/mL and fluorescein in the range of 0.001 to 2.5 mg/mL.

In a particularly preferred embodiment, the present invention provides a pharmaceutical solution comprising water, ketamine hydrochloride in the range of 10 mg/mL to 250 mg/mL, and fluorescein in the range of 0.05 to 1 mg/mL.

In a particularly preferred embodiment, the present invention provides a pharmaceutical solution comprising water, propylene glycol, ketamine hydrochloride in the concentration of 100 mg/mL, and fluorescein in the concentration of 0.15 mg/mL.

An embodiment of the invention is a pharmaceutical composition which includes a colored solution of atropine.

An embodiment of the invention is a pharmaceutical composition which comprises a green solution of atropine.

In another embodiment, the present invention provides a method for the induction of an anticholinergic effect in a mammal which includes administering a colored solution of atropine to the mammal.

In another embodiment, the present invention provides a method for the induction of an anticholinergic effect in a mammal which includes administering a green solution of atropine, wherein said green color is derived from fluorescein and methylene blue.

In another embodiment, the present invention provides a method for the induction of an anticholinergic effect in a mammal which includes administering a green solution of atropine, wherein said green color is derived from fluorescein and indigo carmine.

In a preferred embodiment, the present invention provides a pharmaceutical composition which comprises a solution of water, atropine, fluorescein, and methylene blue.

In a preferred embodiment, the present invention provides a pharmaceutical composition which comprises a solution of water, atropine, fluorescein, and indigo carmine.

In another embodiment, the present invention provides a pharmaceutical composition which comprises a solution of water, atropine, fluorescein, and methylene blue for use as a medicament.

In another embodiment, the present invention provides a pharmaceutical composition which comprises a solution of water, atropine, fluorescein, and indigo carmine for use as a medicament.

In a particularly preferred embodiment, the present invention provides a pharmaceutical solution comprising water, atropine in the range of 0.04 mg/mL to 4.0 mg/mL, methylene blue in the range of 0.001 to 0.05 mg/mL, and fluorescein in the range of 0.001 to 0.1 μg/mL.

In a particularly preferred embodiment, the present invention provides a pharmaceutical solution comprising water, atropine in the range of 0.1 mg/mL to 2.0 mg/mL, methylene blue in the range of 0.002 to 0.02 mg/mL, and fluorescein in the range of 0.002 to 0.02 mg/mL.

In a particularly preferred embodiment, the present invention provides a pharmaceutical solution comprising water, atropine in the concentration of 1 mg/mL, methylene blue in the concentration of 0.003 mg/mL, and fluorescein in the concentration of 0.004 mg/mL.

In a particularly preferred embodiment, the present invention provides a pharmaceutical solution comprising water, atropine in the range of 0.04 mg/mL to 4.0 mg/mL, indigo carmine in the range of 0.001 to 0.05 mg/mL, and fluorescein in the range of 0.001 to 0.4 mg/mL.

In a particularly preferred embodiment, the present invention provides a pharmaceutical solution comprising water, atropine in the range of 0.1 mg/mL to 2.0 mg/mL, indigo carmine in the range of 0.001 to 0.03 mg/mL, and fluorescein in the range of 0.002 to 0.2 mg/mL.

In a particularly preferred embodiment, the present invention provides a pharmaceutical solution comprising water, atropine in the concentration of 1 mg/mL, indigo carmine in the concentration of 0.015 mg/mL and fluorescein in the concentration of 0.006 mg/mL.

An embodiment of the invention is a pharmaceutical composition comprising a colored solution of glycopyrrolate.

An embodiment of the invention is a pharmaceutical composition which comprises a green solution of glycopyrrolate.

In another embodiment, the present invention provides a method for the induction of an anticholinergic effect in a mammal which includes administering a colored solution of glycopyrrolate to the mammal.

In another embodiment, the present invention provides a method for the induction of an anticholinergic effect in a mammal which includes administering a green solution of glycopyrrolate, wherein the green color of said solution is derived from fluorescein and methylene blue.

In another embodiment, the present invention provides a method for the induction of an anticholinergic effect in a mammal which includes administering a green solution of glycopyrrolate, wherein the green color of said solution is derived from fluorescein and indigo carmine.

In a preferred embodiment, the present invention provides a pharmaceutical composition which comprises a solution of water, glycopyrrolate, fluorescein, and methylene blue.

In a preferred embodiment, the present invention provides a pharmaceutical composition which comprises a solution of water, glycopyrrolate, fluorescein, and indigo carmine.

In another embodiment, the present invention provides a pharmaceutical composition which comprises a solution of water, glycopyrrolate, fluorescein, and methylene blue for use as a medicament.

In another embodiment, the present invention provides a pharmaceutical composition which comprises a solution of water, glycopyrrolate, fluorescein, and indigo carmine for use as a medicament.

In a particularly preferred embodiment, the present invention provides a pharmaceutical solution comprising water, glycopyrrolate in the range of 0.002 mg/mL to 2.0 mg/mL, methylene blue in the range of 0.001 to 0.05 mg/mL, and fluorescein in the range of 0.001 to 0.1 mg/mL.

In a particularly preferred embodiment, the present invention provides a pharmaceutical solution comprising water, glycopyrrolate in the range of 0.05 mg/mL to 1.0 mg/mL, methylene blue in the range of 0.002 to 0.02 mg/mL, and fluorescein in the range of 0.002 to 0.02 mg/mL.

In a particularly preferred embodiment, the present invention provides a pharmaceutical solution comprising water, glycopyrrolate in the concentration of 0.2 mg/mL, methylene blue in the concentration of 0.003 mg/mL, and fluorescein in the concentration of 0.004 mg/mL.

In a particularly preferred embodiment, the present invention provides a pharmaceutical solution comprising water, glycopyrrolate in the range of 0.002 mg/mL to 2.0 mg/mL, indigo carmine in the range of 0.001 to 0.05 mg/mL, and fluorescein in the range of 0.001 to 0.4 mg/mL.

In a particularly preferred embodiment, the present invention provides a pharmaceutical solution comprising water, glycopyrrolate in the range of 0.001 mg/mL to 1.0 mg/mL, indigo carmine in the range of 0.001 to 0.03 mg/mL, and fluorescein in the range of 0.002 to 0.2 mg/mL.

In a particularly preferred embodiment, the present invention provides a pharmaceutical solution comprising water, glycopyrrolate in the concentration of 0.2 mg/mL, indigo carmine in the concentration of 0.015 mg/mL and fluorescein in the concentration of 0.006 mg/mL.

An embodiment of the invention is a pharmaceutical composition comprising a colored solution of gantacurium.

An embodiment of the invention is a pharmaceutical solution which comprises a bright orange solution of gantacurium.

An embodiment of the invention is a pharmaceutical solution which comprises a bright orange solution of gantacurium, wherein said orange color is derived from fluorescein.

An embodiment of the invention is a pharmaceutical solution which comprises a bright orange solution aqueous of gantacurium, wherein said orange color is derived from fluorescein.

In one embodiment, the present invention provides a pharmaceutical solution which includes a therapeutically effective amount of a colored solution of gantacurium for eliciting a muscle relaxant response in a mammal.

In another embodiment, the present invention provides a method for eliciting a muscle relaxant response in a mammal which includes administering a therapeutically effective amount of a colored solution of gantacurium to the mammal that is sufficient to elicit a muscle relaxant response.

In another embodiment, the present invention provides a method for eliciting a muscle relaxant response in a mammal which includes administering a therapeutically effective amount of a bright orange aqueous solution of gantacurium, wherein the bright orange color of said solution is derived from fluorescein.

In a particularly preferred embodiment, the present invention provides a pharmaceutical solution comprising water, gantacurium in the range of 0.1 mg/mL to 250 mg/mL and fluorescein in the range of 7 to 250 mg/mL.

In a particularly preferred embodiment, the present invention provides a pharmaceutical solution comprising water, gantacurium in the range of 1 mg/mL to 50 mg/mL, and fluorescein in the range of 7 to 100 mg/mL.

An embodiment of the invention is a pharmaceutical composition comprising a colored solution of mivacurium.

An embodiment of the invention is a pharmaceutical solution which comprises a bright orange solution of mivacurium.

An embodiment of the invention is a pharmaceutical solution which comprises a bright orange solution of mivacurium, wherein said orange color is derived from fluorescein.

An embodiment of the invention is a pharmaceutical solution which comprises a bright orange solution aqueous of mivacurium, wherein said orange color is derived from fluorescein.

In one embodiment, the present invention provides a pharmaceutical solution which includes a therapeutically effective amount of a colored solution of mivacurium for eliciting a muscle relaxant response in a mammal.

In another embodiment, the present invention provides a method for eliciting a muscle relaxant response in a mammal which includes administering a therapeutically effective amount of a colored solution of mivacurium to the mammal that is sufficient to elicit a muscle relaxant response.

In another embodiment, the present invention provides a method for eliciting a muscle relaxant response in a mammal which includes administering a therapeutically effective amount of a bright orange aqueous solution of mivacurium, wherein the bright orange color of said solution is derived from fluorescein.

In a particularly preferred embodiment, the present invention provides a pharmaceutical solution comprising water, mivacurium in the range of 0.1 mg/mL to 250 mg/mL and fluorescein in the range of 7 to 250 mg/mL.

In a particularly preferred embodiment, the present invention provides a pharmaceutical solution comprising water, mivacurium in the range of 1 mg/mL to 50 mg/mL, and fluorescein in the range of 7 to 100 mg/mL.

### DEFINITIONS

As used herein, the term “about” is intended to mean+/−15%.

As used herein, the term “anticholinergic” is intended to mean a pharmaceutical which antagonizes the muscarinic effects of acetylcholine by competing for the same receptors as are normally occupied by the neurotransmitter. Examples of anticholinergic pharmaceuticals include atropine and glycopyrrolate.

As used herein the term “atracurium besylate” refers to the di(benzenesulfonate) salt of 2,2′-(3,11-dioxo-4,10-dioxamidecamethylene)bis(1,2,3,4-tetrahydro-6,7-dimethoxy-2-methyl-1-veratrylisoquinolinium), as shown in Formula 1.

As used herein the term “bright orange” refers to a color characterized by the reflection of light of one or more wavelengths, predominantly light of a wavelength between 570 and 650 nm. Examples include, but are not limited to: Pantone 881, and aqueous solutions of fluorescein within the range of about 25 mg/mL to 10 mg/mL.

As used herein the term “color coding” is the systematic application of a standardized color system which allows users to classify and identify both classes of drugs and individual drug products. Such a system would enable users to match a color to a standardized pharmaceutical function.

As used herein the term “green” refers to a color characterized by the reflection of light of one or more wavelengths, predominantly light of a wavelength between 530 and 490 nm. Examples include, but are not limited to: pantone 367, aqueous solutions of fluorescein and methylene blue, and aqueous solutions of fluorescein and indigo carmine.

As used herein, the term “hypnotic” is intended to mean a pharmaceutical which induces sleep, or relieves anxiety, or relieves seizures. Examples of hypnotic drugs include, but are not limited to diazepam, lorazepam, and midazolam.

As used herein, the term “induction agent” is intended to mean a pharmaceutical which is used for the induction and maintenance of general anesthesia, and/or the induction and maintenance of sedation. Examples of induction agents include etomidate, ketamine, propofol, and fospropofol disodium.

As used herein the term “mammal” refers to any of various warm-blooded vertebrate animals, including humans, characterized by a covering of hair on the skin and, in the female, milk-producing mammary glands for nourishing the young. Mammals include, for example, humans, as well as pet animals such as dogs and cats, laboratory animals, such as rats and mice, and farm animals, such as horses and cows.

As used herein, the term “muscle relaxant” is intended to mean a pharmaceutical which is used as an adjunct to general anesthesia, to facilitate mechanical ventilation, endotracheal intubation, and to provide skeletal muscle relaxation during surgery or maniplulative procedures. Examples of muscle relaxants include atracurium, rocuronium, cisatracurium, succinylcholine, pancuronium, vecuronium, gantacurium, and mivacurium.

As used herein the term “orange” refers to a color characterized by the reflection of light of one or more wavelengths, predominantly light of a wavelength between 570 and 650 nm. Examples include, but are not limited to: orange 151, and aqueous solutions of fluorescein within the range of about 1 mg/mL to 10 mg/mL.

As used herein the term “Pantone” refers to the company which has developed an international numeric standard for referencing colors and related color systems. This numeric identification of a color enables accurate communication between designers, manufacturers and end users.

As used herein the term “pharmaceutically acceptable anion” refers to any one of a group of inorganic or organic anions known in the art which balance the charge of the cationic drug. Examples of such anions include the following: chloride, bromide, sulfate, phosphate, methanesulfonate, formate, acetate, trifluoroacetate, citrate, fumarate, malate, tartate, succinate, and salicylate.

As used herein the term “pharmaceutically acceptable salt” refers to any one of a group of inorganic or organic acids known in the art which are combined with the free base form of a drug. Examples of such acids include the following: hydrochloric acid, sulfuric acid, phosphoric acid, methanesulfonic acid, formic acid, acetic acid, citric acid, fumaric acid, maleic acid, tartaric acid, succinic acid, and salicylic acid.

As used herein the term “pharmaceutical composition” refers to an injectable formulation, including injectable solutions, injectable emulsions, and powders which become injectable solutions upon the addition of water or another pharmaceutically acceptable diluent.

As used herein the term “rocuronium” refers to 1-(17β-(acetyloxy)-3α-hydroxy-2β-(4-morpholinyl)-5a-androstan-16β-yl-1-(2-propenyl)pyrrolidinium together with any pharmaceutically acceptable anion.

As used herein the term “rocuronium bromide” refers to 1-[17β-(acetyloxy)-3α-hydroxy-2β-(4-morpholinyl)-5α-androstan-16β-yl]-1-(2-propenyl)pyrrolidinium together with one bromide anion, as shown in Formula 2.

As used herein the term “vecuronium” refers to (+)-1-(3,17-diacetoxy-2-piperidino-5-androstan-16-yl)-1-methylpiperidinium together with any pharmaceutically acceptable anion.

As used herein the term “vecuronium bromide” refers to (+)-1-(3,17-diacetoxy-2-piperidino-5-androstan-16-yl)-1-methylpiperidinium together with one bromide anion, as shown in Formula 3.

As used herein the term “cisatracurium besylate” refers to the di(benzenesulfonate) salt of [1R-[1α,2α(1′R*,2′R*)]]-2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methylisoquinolinium], as shown in Formula 4. It is understood by those skilled in the art that cisatracurium besylate is one of 10 isomers and constitutes approximately 15% of said mixture.

As used herein the term “pancuronium” refers to 3α,17β-diacetoxy-5α-androstan-2β,16β-ylene bis[1-methylpiperidinium] together with any pharmaceutically acceptable anion.

As used herein the term “pancuronium bromide” refers to 3α,17β-diacetoxy-5α-androstan-2β,16β-ylene bis[1-methylpiperidinium] together with two bromide anions, as shown in Formula 5.

As used herein the term “succinylcholine” refers to 2,2′-[(1,4-dioxo-1,4-butanediyl)bis(oxy)]bis[N,N,N-trimethylethanaminium] together with any pharmaceutically acceptable anion.

As used herein the term “succinylcholine dichloride” refers to 2,2′-[(1,4-dioxo-1,4-butanediyl)bis(oxy)]bis[N,N,N-trimethylethanaminium] together with two chloride anions, as shown in Formula 6.

As used herein the term “gantacurium” is intended to mean (1R,2S)-2-(3-{[(2Z)-2-chloro-4-{3-[(1S,2R)-6,7-dimethoxy-2-methyl-1-(3,4,5-trimethoxyphenyl)-1,2,3,4-tetrahydroisoquinolinium-2-yl]propoxy}-4-oxobut-2-enoyl]oxy}propyl)-6,7-dimethoxy-2-methyl-1-[(3,4,5-trimethoxyphenyl)methyl]-1,2,3,4-tetrahydroisoquinolinium dichloride. Included in the definition are (1R,2S)-2-(3-{[(2Z)-2-chloro-4-{3-[(1S,2R)-6,7-dimethoxy-2-methyl-1-(3,4,5-trimethoxyphenyl)-1,2,3,4-tetrahydroisoquinolinium-2-yl]propoxy}-4-oxobut-2-enoyl]oxy}propyl)-6,7-dimethoxy-2-methyl-1-[(3,4,5-trimethoxyphenyl)methyl]-1,2,3,4-tetrahydroisoquinolinium cations paired with pharmaceutically acceptable anions other than chloride.

As used herein, the term “mivacuriur” is intended to mean [R—[R*,R*-(E))]]-2,2′-[(1,8-dioxo-4-octene-1,8-diyl)bis(oxy-3,1-propanediyl)]bis[1,2,3,4-tetrahydro-6,7-dimethoxy-2-methyl-1-[(3,4,5-trimethoxyphenyl)methyl]isoquinolinium]dichloride. Included in the definition are [R—[R*,R*-(E))]-2,2′-((1,8-dioxo-4-octene-1,8-diyl)bis(oxy-3,1-propanediyl)]bis[1,2,3,4-tetrahydro-6,7-dimethoxy-2-methyl-1-[(3,4,5-trimethoxyphenyl)methyl]isoquinolinium] cations paired with pharmaceutically acceptable anions other than chloride.

As used herein the term “lorazepam” refers to 7-chloro-5-(o-chlorophenyl)-1,3-dihydro-3-hydroxy-2H-1,4-benzodiazepin-2-one, as shown in formula 7.

As used herein the term “midazolam hydrochloride” refers to the hydrochloride salt of 8-chloro-6-(2-fluorophenyl)-1-methyl-4H-imidazo(1,5-a)(1,4)benzodiazepine.

As used herein the term “diazepam” refers to 7-chloro-1,3-dihydro-1-methyl-5-phenyl-2H-1,4-benzodiazepin-2-one, as shown in formula 9.

As used herein the term “etomidate” refers to (R)-(+)-ethyl-1-(1-phenylethyl)-1H-imidazole-5-carboxylate, as shown in Formula 10.

As used herein the term “propofol” refers to 2,6-diisopropylphenol, as shown in Formula 11.

As used herein the term “fospropofol disodium” refers to disodium (2,6-diisopropylphenoxy)methyl phosphate, as shown in Formula 12.

As used herein the term “ketamine” refers to 2-(2-chlorophenyl)-2-(methylamino)-cyclohexanone, which may be in either its free base form or that of a pharmaceutically acceptable salt, such as, for example: ketamine hydrochloride.

As used herein the term “atropine” refers to atropine sulfate monohydrate.

As used herein the term “glycopyrrolate” refers to 3-[(cyclopentylhydroxyphenylacetyl)oxy]-1,1-dimethylpyrrolidinium bromide, as shown in Formula 15.

As used herein the term “therapeutically effective amount of a colored solution of atracurium besylate” refers to that amount effective to elicit a muscle relaxant response. The amount will be the same amount referred to by the term “therapeutically effective amount of a bright orange solution of atracurium besylate.” For example, a colored solution of atracurium besylate can achieve skeletal muscle relaxation in an anesthetized patient. Preferably, said solution is administered in an amount that limits the most common side effects such as allergic reactions, tachycardia, decreased blood pressure and seizures.

As used herein the term “therapeutically effective amount of a colored solution of rocuronium bromide” refers to that amount effective to elicit a muscle relaxant response. The amount will be the same amount referred to by the term “therapeutically effective amount of a bright orange solution of rocuronium bromide.” For example, a colored solution of rocuronium can achieve skeletal muscle relaxation in an anesthetized patient. Preferably, said solution is administered in an amount that limits the most common side effects such as allergic reactions.

As used herein the term “a therapeutically effective amount of a bright orange solution of vecuronium bromide” refers to that amount effective to elicit a muscle relaxant response. For example, a bright orange solution of vecuronium can achieve skeletal muscle relaxation in an anesthetized patient. Preferably, said solution is administered in an amount that limits the most common side effects such as allergic reactions or accumulation of a large unmetabolized portion of the dose.

As used herein the term “therapeutically effective amount of a colored solution of cisatracurium besylate” refers to that amount effective to elicit a muscle relaxant response. The amount will be the same amount referred to by the term “therapeutically effective amount of a bright orange solution of cisatracurium besylate.” For example, a colored solution of cisatracurium besylate can achieve skeletal muscle relaxation in an anesthetized patient. Preferably, said solution is administered in an amount that limits the most common side effects such as allergic reactions.

As used herein the term “therapeutically effective amount of a colored solution of pancuronium bromide” refers to that amount effective to elicit a muscle relaxant response. The amount will be the same amount referred to by the term “therapeutically effective amount of a bright orange solution of pancuronium bromide.” For example, a colored solution of pancuronium can achieve skeletal muscle relaxation in an anesthetized patient. Preferably, said solution is administered in an amount that limits the most common side effects such as increased heart rate and blood pressure.

As used herein the term “therapeutically effective amount of a colored solution of succinylcholine” refers to that amount effective to elicit a muscle relaxant response. The amount will be the same amount referred to by the term “therapeutically effective amount of a bright orange solution of succinylcholine.” For example, a colored solution of succinylcholine can achieve skeletal muscle relaxation in an anesthetized patient. Preferably, said solution is administered in an amount that limits the most common side effects such as hyperkalemia, cardiac dysrhythmias, myoglobinuria, increased intraocular and increased intragastric pressures, trismus, myalgia, fasciculations, allergic reaction, and malignant hyperthermia.

As used herein the term “therapeutically effective amount of a colored solution of gantacurium” refers to that amount effective to elicit a muscle relaxant response. The amount will be the same amount referred to by the term “therapeutically effective amount of a bright orange solution of gantacurium dichloride.” For example, a colored solution of gantacurium can achieve skeletal muscle relaxation in an anesthetized patient. Preferably, said solution is administered in an amount that limits the most common side effects such as hypotension.

As used herein the term “therapeutically effective amount of a colored solution of mivacurium” refers to that amount effective to elicit a muscle relaxant response. The amount will be the same amount referred to by the term “therapeutically effective amount of a bright orange solution of mivacurium dichloride.” For example, a colored solution of mivacurium can achieve skeletal muscle relaxation in an anesthetized patient. Preferably, said solution is administered in an amount that limits the most common side effects such as cutaneous flushing around the face, neck and/or chest, and hypotension.

As used herein the term “therapeutically effective amount of a colored solution of etomidate” refers to that amount of a colored solution of etomidate effective for the induction of general anesthesia. The amount will be the same amount referred to by the term “therapeutically effective amount of a yellow solution of etomidate.” For example, a colored solution of etomidate can induce general anesthesia. Preferably, said solution is administered in an amount that limits the most common side effects such as transient venous pain, transient skeletal muscle movements, seizures, apnea and adrenal gland suppression.

As used herein the term “therapeutically effective amount of a colored solution of propofol” refers to that amount of a colored solution or emulsion of propofol effective for any of the common uses of propofol. The amount will be the same amount referred to by the term “therapeutically effective amount of a yellow solution of propofol.” Such uses include, for example: the induction and maintenance of general anesthesia, and the induction and maintenance of sedation. For example, a colored solution or emulsion of propofol can induce general anesthesia. Preferably, said solution or emulsion is administered in an amount that limits the most common side effects such as transient venous pain, hypotension, bradycardia, asystole, and apnea.

As used herein the term “therapeutically effective amount of a colored solution of fospropofol disodium” refers to that amount of a colored solution of fospropofol disodium effective for any of the common uses of fospropofol disodium. The amount will be the same amount referred to by the term “therapeutically effective amount of a yellow solution of fospropofol disodium” and said amount will be the same amount referred to by the term “therapeutically effective amount of an orange solution of fospropofol disodium.” Such uses include, for example: the induction and maintenance of general anesthesia, and the induction and maintenance of sedation. For example, a colored solution of fospropofol disodium can induce general anesthesia. Preferably, said solution is administered in an amount that limits the most common side effects such as transient venous pain, hypotension, bradycardia, asystole, and apnea.

As used herein the term “therapeutically effective amount of a colored solution of ketamine” refers to that amount of colored solution of ketamine hydrochloride effective for the induction of general anesthesia. The amount will be the same amount referred to by the term “therapeutically effective amount of a yellow aqueous solution of ketamine.” For example, a colored solution of ketamine hydrochloride can induce general anesthesia. Preferably, said solution is administered in an amount that limits the most common side effects such as emergence delirium, increased heart rate, contractility, blood pressure and increased intracranial pressure.

As used herein the term “yellow” refers to a color characterized by the reflection of light of one or more wavelengths, predominantly light of a wavelength between 590 and 540 nm. Examples include, but are not limited to: aqueous solutions or emulsions of fluorescein within the range of about 0.02 mg/mL to 1.0 mg/mL.

### EXAMPLES

The following examples are for illustrative purposes only, and are in no way meant to limit the invention.

### Example 1

A sterile aqueous solution, wherein each mL contains atracurium besylate (10 mg), and fluorescein (10 mg), wherein the pH of said solution is adjusted to 3.5 with benzenesulfonic acid.

### Example 2

A sterile aqueous solution, wherein each mL contains atracurium besylate (10 mg), benzyl alcohol (10 mg), and fluorescein (10 mg), wherein the pH of said solution is adjusted to 3.5 with benzenesulfonic acid.

### Example 3

A sterile aqueous solution, wherein each mL contains rocuronium bromide (10 mg), sodium acetate, trihydrate (2 mg), sodium chloride (3.3 mg), and fluorescein (50 mg), wherein the pH of said solution is adjusted to 4.0 with acetic acid.

### Example 4

A sterile aqueous solution, wherein each mL contains rocuronium bromide (10 mg), sodium acetate, trihydrate (2 mg), sodium chloride (3.3 mg), and fluorescein (15 mg), wherein the pH of said solution is adjusted to 4.0 with acetic acid.

### Example 5

A sterile aqueous solution, wherein each mL contains rocuronium bromide (10 mg), sodium acetate, trihydrate (2 mg), sodium chloride (3.3 mg), and fluorescein (7 mg), wherein the pH of said solution is adjusted to 4.0 with acetic acid.

### Example 6

A powder comprising vecuronium bromide (10 mg), citric acid (20.75 mg), dibasic sodium phosphate (16.25 mg), mannitol (97 mg), fluorescein (100 mg), and phosphoric acid and sodium hydroxide so that upon the addition of 10 mL water, the pH of the resulting solution will be 4.

### Example 7

A sterile aqueous solution, wherein each mL contains cisatracurium besylate (10 mg), and fluorescein (10 mg), wherein the pH of said solution is adjusted to 3.5 with benzenesulfonic acid.

### Example 8

A sterile aqueous solution, wherein each mL contains cisatracurium besylate (10 mg), benzyl alcohol (10 mg), and fluorescein (10 mg), wherein the pH of said solution is adjusted to 3.5 with benzenesulfonic acid.

### Example 9

A sterile aqueous solution, wherein each mL contains cisatracurium besylate equivalent to 10 mg cisatracurium, and fluorescein (10 mg), wherein the pH of said solution is adjusted to 3.5 with benzenesulfonic acid.

### Example 10

A sterile aqueous solution, wherein each mL contains cisatracurium besylate equivalent to 10 mg cisatracurium, benzyl alcohol (10 mg), and fluorescein (10 mg), wherein the pH of said solution is adjusted to 3.5 with benzenesulfonic acid.

### Example 11

A sterile aqueous solution, wherein each mL contains cisatracurium besylate equivalent to 2 mg cisatracurium, and fluorescein (10 mg), wherein the pH of said solution is adjusted to 3.5 with benzenesulfonic acid.

### Example 12

A sterile aqueous solution, wherein each mL contains cisatracurium besylate equivalent to 2 mg cisatracurium, benzyl alcohol (10 mg), and fluorescein (10 mg), wherein the pH of said solution is adjusted to 3.5 with benzenesulfonic acid.

### Example 13

A sterile aqueous solution, wherein each mL contains pancuronium bromide (1 mg), sodium acetate anhydrous (1.2 mg), benzyl alcohol (10 mg), sodium chloride for tonicity about (5 mg), and fluorescein (15 mg), wherein the pH of said solution is adjusted to 4.0 with acetic acid.

### Example 14

A sterile aqueous solution, wherein each mL contains pancuronium bromide (2 mg), sodium acetate anhydrous (1.2 mg), benzyl alcohol (10 mg), sodium chloride for tonicity about (5 mg), and fluorescein (15 mg), wherein the pH of said solution is adjusted to 4.0 with acetic acid.

### Example 15

A sterile aqueous solution, wherein each mL contains pancuronium bromide (1 mg), sodium acetate anhydrous (1.2 mg), benzyl alcohol (10 mg), sodium chloride for tonicity about (5 mg), and fluorescein (7 mg), wherein the pH of said solution is adjusted to 4.0 with acetic acid.

### Example 16

A sterile aqueous solution, wherein each mL contains pancuronium bromide (2 mg), sodium acetate anhydrous (1.2 mg), benzyl alcohol (10 mg), sodium chloride for tonicity about (5 mg), and fluorescein (7 mg), wherein the pH of said solution is adjusted to 4.0 with acetic acid.

### Example 17

A sterile aqueous solution, wherein each mL contains pancuronium bromide (10 mg), sodium acetate, trihydrate (2 mg), sodium chloride (3.3 mg), and fluorescein (15 mg), wherein the pH of said solution is adjusted to 4.0 with acetic acid.

### Example 18

A sterile aqueous solution, wherein each mL contains pancuronium bromide (10 mg), sodium acetate, trihydrate (2 mg), sodium chloride (3.3 mg), and fluorescein (7 mg), wherein the pH of said solution is adjusted to 4.0 with acetic acid.

### Example 19

A pharmaceutical composition comprising a sterile aqueous solution, wherein each mL contains succinylcholine dichloride (20 mg), sodium chloride (4.5 mg), and fluorescein (10 mg), wherein the pH of said solution is adjusted to 3.5 with hydrochloric acid.

### Example 20

A pharmaceutical composition comprising a sterile aqueous solution, wherein each mL contains succinylcholine dichloride (20 mg), sodium chloride (4.5 mg), methylparaben (0.1% w/w) and fluorescein (10 mg), wherein the pH of said solution is adjusted to 3.5 with hydrochloric acid.

### Example 21

A pharmaceutical composition comprising a sterile aqueous solution, wherein each mL contains succinylcholine dichloride (50 mg), sodium chloride (4.5 mg), and fluorescein (10 mg), wherein the pH of said solution is adjusted to 3.5 with hydrochloric acid.

### Example 22

A pharmaceutical composition comprising a sterile aqueous solution, wherein each mL contains succinylcholine dichloride (50 mg), sodium chloride (4.5 mg), methylparaben (0.1% w/w) and fluorescein (10 mg), wherein the pH of said solution is adjusted to 3.5 with hydrochloric acid.

### Example 23

A pharmaceutical composition comprising a sterile aqueous solution, wherein each mL contains succinylcholine dichloride (100 mg), sodium chloride (4.5 mg), and fluorescein (10 mg), wherein the pH of said solution is adjusted to 3.5 with hydrochloric acid.

### Example 24

A pharmaceutical composition comprising a sterile aqueous solution, wherein each mL contains succinylcholine dichloride (100 mg), sodium chloride (4.5 mg), methylparaben (0.1% w/w) and fluorescein (10 mg), wherein the pH of said solution is adjusted to 3.5 with hydrochloric acid.

### Example 25

A pharmaceutical composition comprising a sterile aqueous solution, wherein each mL contains succinylcholine dichloride (20 mg), sodium chloride (4.5 mg), methylparaben (0.18% w/w), propylparaben (0.02% w/w), and fluorescein (10 mg), wherein the pH of said solution is adjusted to 3.5 with hydrochloric acid.

### Example 26

A pharmaceutical composition comprising a sterile aqueous solution, wherein each mL contains succinylcholine dichloride (50 mg), sodium chloride (4.5 mg), methylparaben (0.18% w/w), propylparaben (0.02% w/w), and fluorescein (10 mg), wherein the pH of said solution is adjusted to 3.5 with hydrochloric acid.

### Example 27

A pharmaceutical composition comprising a sterile aqueous solution, wherein each mL contains succinylcholine dichloride (100 mg), sodium chloride (4.5 mg), methylparaben (0.18% w/w), propylparaben (0.02% w/w), and fluorescein (10 mg), wherein the pH of said solution is adjusted to 3.5 with hydrochloric acid.

### Example 28

A pharmaceutical solution comprising propylene glycol (80% v/v), polyethylene glycol (18% v/v), and benzyl alcohol (2% v/v), said solution further comprising lorazepam in the concentration of 2 mg/mL, and fluorescein in the concentration of 2.5 mg/mL.

### Example 29

A sterile aqueous solution, wherein each mL contains midazolam hydrochloride (1 mg), sodium chloride (0.8% w/v), disodium edentate (0.01% w/v), benzyl alcohol (1% w/v), and fluorescein (2.5 mg), wherein the pH of said solution is adjusted to 3 with hydrochloric acid.

### Example 30

A sterile aqueous solution, wherein each mL contains midazolam hydrochloride (5 mg), sodium chloride (0.8% w/v), disodium edentate (0.01% w/v), benzyl alcohol (1% w/v), and fluorescein (2.5 mg), wherein the pH of said solution is adjusted to 3 with hydrochloric acid.

### Example 31

A solution comprising propylene glycol (80% v/v), polyethylene glycol (18% v/v), and benzyl alcohol (2% v/v), said solution further comprising diazepam in the concentration of 4 mg/mL, and fluorescein in the concentration of 2.5 mg/mL.

### Example 32

A solution comprising water for injection, diazepam (5 mg/mL) propylene glycol (40% w/v), ethyl alcohol (10% w/v), fluorescein (2.5 mg/mL), sodium benzoate and benzoic acid sufficient to bring the pH to 6.5 (5% w/v), and benzyl alcohol (1.5% w/v).

### Example 33

An emulsion comprising diazepam (5 mg/mL), fractionated soybean oil (150 mg/mL), diacetylated monoglycerides (50 mg/mL), fractionated egg yolk phospholipids (12 mg/mL), glycerin (22.0 mg/mL), fluorescein (2.5 mg/mL), water for injection, and sodium hydroxide to adjust pH to approximately 8.

### Example 34

A sterile aqueous solution, wherein each mL contains etomidate (2 mg), propylene glycol 35% (v/v) and fluorescein (0.15 mg).

### Example 35

A sterile aqueous solution, wherein each mL contains ketamine hydrochloride (100 mg), benzethonium chloride (0.1 mg), and fluorescein (0.15 mg).

### Example 36

A pharmaceutical solution comprising water, atropine (0.4 mg/mL), methylparaben (0.1% w/v), methylene blue (0.003 mg/mL), and fluorescein (0.004 mg/mL), wherein the pH of said solution is adjusted to 4.5 with sulfuric acid.

### Example 37

A pharmaceutical solution comprising water, atropine (0.4 mg/ml), methylparaben (0.1% w/v), indigo carmine (0.015 mg/ml), and fluorescein (0.006 mg/ml), wherein the pH of said solution is adjusted to 4.5 with sulfuric acid.

### Example 38

A pharmaceutical solution comprising water, atropine (0.5 mg/ml), methylparaben (0.1% w/v), methylene blue (0.003 mg/ml), and fluorescein (0.004 mg/ml), wherein the pH of said solution is adjusted to 4.5 with sulfuric acid.

### Example 39

A pharmaceutical solution comprising water, atropine (0.5 mg/ml), methylparaben (0.1% w/v), indigo carmine (0.015 mg/ml), and fluorescein (0.006 mg/ml), wherein the pH of said solution is adjusted to 4.5 with sulfuric acid.

### Example 40

A pharmaceutical solution comprising water, atropine (1 mg/ml), methylparaben (0.1% w/v), methylene blue (0.003 mg/ml), and fluorescein (0.004 mg/ml), wherein the pH of said solution is adjusted to 4.5 with sulfuric acid.

### Example 41

A pharmaceutical solution comprising water, atropine (1 mg/ml), methylparaben (0.1% w/v), indigo carmine (0.015 mg/ml), and fluorescein (0.006 mg/ml), wherein the pH of said solution is adjusted to 4.5 with sulfuric acid.

### Example 42

A pharmaceutical solution comprising water, atropine (0.1 mg/ml), sodium chloride (9 mg/ml), indigo carmine (0.015 mg/ml), and fluorescein (0.006 mg/ml), wherein the pH of said solution is adjusted to 4.2 with sulfuric acid.

### Example 43

A pharmaceutical solution comprising water, atropine (0.05 mg/ml), sodium chloride (9 mg/ml), indigo carmine (0.015 mg/ml), and fluorescein (0.006 mg/ml), wherein the pH of said solution is adjusted to 4.2 with sulfuric acid.

### Example 44

A pharmaceutical solution comprising water, atropine (0.1 mg/ml), sodium chloride (9 mg/ml), methylene blue (0.003 mg/ml), and fluorescein (0.004 mg/ml), wherein the pH of said solution is adjusted to 4.2 with sulfuric acid.

### Example 45

A pharmaceutical solution comprising water, atropine (0.05 mg/ml), sodium chloride (9 mg/ml), methylene blue (0.003 mg/ml), and fluorescein (0.004 mg/ml), wherein the pH of said solution is adjusted to 4.2 with sulfuric acid.

### Example 46

A sterile aqueous solution, wherein each mL contains water, glycopyrrolate (0.2 mg), benzyl alcohol (0.9% w/v), methylene blue (0.003 mg), and fluorescein (0.004 mg).

### Example 47

A sterile aqueous solution, wherein each mL contains water, glycopyrrolate (0.2 mg), benzyl alcohol (0.9% w/v), indigo carmine (0.015 mg), and fluorescein (0.006 mg).

In the above examples, fluorescein is intended to mean the disodium salt of 3′,6′-dihydroxy-spiro[isobenzofuran-1(3H), 9′-[9H]xanthen]-3-one. Those of ordinary skill in the art will appreciate that other cations having the characteristics of sodium can be used without departing from the spirit and scope of the present invention.

In the above examples, methylene blue is intended to mean 3,7-bis(dimethylamino)phenazathionium chloride. Those of ordinary skill in the art will appreciate that other anions having the characteristics of chloride can be used without departing from the spirit and scope of the present invention.

In the above examples, indigo carmine is intended to mean the disodium salt of 5,5′-indigodisulfonic acid. Those of ordinary skill in the art will appreciate that other cations having the characteristics of sodium can be used without departing from the spirit and scope of the present invention.

In the above examples, atracurium besylate is intended to mean the di(benzenesulfonate) salt of 2,2′-(3,11-dioxo-4,10-dioxamidecamethylene) bis(1,2,3,4-tetrahydro-6,7-dimethoxy-2-methyl-1-veratrylisoquinolinium). Those of ordinary skill in the art will appreciate that other pharmaceutically acceptable anions having the characteristics of benzenesulfonate can be used without departing from the spirit and scope of the present invention.

In the above examples, cisatracurium besylate is intended to mean the di(benzenesulfonate) salt of 2,2′-(3,11-dioxo-4,10-dioxamidecamethylene) bis(1,2,3,4-tetrahydro-6,7-dimethoxy-2-methyl-1-veratrylisoquinolinium). Those of ordinary skill in the art will appreciate that other pharmaceutically acceptable anions having the characteristics of benzenesulfonate can be used without departing from the spirit and scope of the present invention.

It is well known in the art that the free base of midazolam may be paired with any number of acids to produce a pharmaceutically acceptable salt. Such variations of colored injectable formulations are considered by the inventor to be within the scope of the invention, as are colored injectable free base emulsions of midazolam.

It is known to those skilled in the art that ketamine has an asymmetric carbon atom, and may be prepared in homochiral or racemic form. Ketamine may also be prepared in any mixture homochiral and racemic forms with enantiomeric excess ranging from 0% to 99.99%. Such variations are considered to be within the spirit and scope of the invention.

In the above examples, fospropofol disodium is intended to mean the disodium salt of (2,6-diisopropylphenoxy)methyl phosphate. Those of ordinary skill in the art will appreciate that other cations having the characteristics of sodium can be used without departing from the spirit and scope of the present invention.

It is known to those skilled in the art that atropine is isolated as a mixture of enantiomers derived from the asymmetric carbon atom between the carboxyl and phenyl groups. It is recognized that colored solutions of atropine may be prepared in homochiral or racemic form. Colored solutions of atropine may also be prepared in any mixture of homochiral and racemic forms with enantiomeric excess ranging from 0% to 99.99%. Such variations are considered to be within the spirit and scope of the invention.

In the above examples glycopyrrolate is intended to mean 3-[(cyclopentylhydroxyphenylacetyl)oxy]-1,1-dimethylpyrrolidinium bromide. Those of ordinary skill in the art will appreciate that other anions having the characteristics of bromide can be used without departing from the spirit and scope of the present invention.

It is known to those skilled in the art that glycopyrrolate is a mixture of enantiomers. It is recognized that colored solutions of glycopyrrolate may be prepared in homochiral or racemic form. Colored solutions of glycopyrrolate may also be prepared in any mixture of homochiral and racemic forms with enantiomeric excess ranging from 0% to 99.99%. Such variations are considered to be within the spirit and scope of the invention.

Colored atracurium besylate solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored rocuronium solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored vecuronium powders may be diluted with sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored cisatracurium besylate solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored pancuronium solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water. A preferred formulation includes sodium chloride for isotonicity. A preferred buffer is acetic acid with sodium acetate.

Colored succinylcholine solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored diazepam solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates may also be added. The intravenous preparation can be enclosed in ampules, disposable syringes or multiple dose vials made of glass or plastic. Preferred diluents are propylene glycol, polyethylene glycol, soybean oil, water, and ethyl alcohol; a preferred antibacterial agent is benzyl alcohol.

Colored gantacurium solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored mivacurium solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored lorazepam solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates may also be added. The intravenous preparation can be enclosed in ampules, disposable syringes or multiple dose vials made of glass or plastic. Preferred diluents are propylene glycol, polyethylene glycol, and a preferred antibacterial agent is benzyl alcohol.

Colored midazolam hydrochloride solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored etomidate solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. Preferred diluents are water and propylene glycol.

Colored propofol solutions or emulsions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. Preferred diluents are water and soybean oil.

Colored fospropofol disodium solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored ketamine solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored atropine solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Colored glycopyrrolate solutions may also include sterile diluents such as water for injection, saline solution, fixed oils, polyethylene glycols, glycerine, propylene glycol or other synthetic solvents. Intravenous formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The intravenous preparation can be enclosed in ampules, disposable syringes or multiple dose vials made of glass or plastic. A preferred diluent is water.

Atracurium besylate solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Rocuronium solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

It is known in the art that vecuronium bromide is commercially available as a dry powder. In common practice a 10 mg vial of vecuronium bromide is diluted with 10 mL of bacteriostatic water for injection, or 10 mL of compatible diluent to obtain a solution containing 1 mg/mL vecuronium bromide. Compatible diluents include: 0.9% sodium chloride injection USP, 5% dextrose injection USP, 5% dextrose and 0.9% sodium chloride injection USP, and sterile water for injection USP, and lactated ringer's injection USP.

Vecuronium solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid. The fluoroscein may be added by any means known in the art. For example, the fluoroscein may be included in the dry vecuronium powder, or it may be added as a solution at the time the vecuronium powder is dissolved.

Cisatracurium besylate solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Pancuronium solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Succinylcholine solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Gantacurium solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Mivacurium solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Diazepam solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Lorazepam solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Midazolam hydrochloride solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Etomidate solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Propofol compositions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Fospropofol disodium compositions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Ketamine solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid.

Atropine solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid. Preferred dyes are fluorescein combined with methylene blue, and fluorescein combined with indigo carmine.

Glycopyrrolate solutions may be colored with fluorescein alone or combined with any of the dyes known in the art. Said dyes include, but are not limited to: FD&C blue #1, FD&C blue #2, methylene blue, indigo carmine, FD&C green #3, FD&C green #5, FD&C red #3, FD&C red #28, FD&C red #33, FD&C red #40, FD&C yellow #5, FD&C yellow #6, FD&C yellow #10, riboflavin, panthotenic acid, and folic acid. Preferred dyes are fluorescein combined with methylene blue, and fluorescein combined with indigo carmine.

It is well known in the art that solutions of atracurium besylate should be stored in a refrigerator at 2 to 8° C., as well as protected from light.

It is well known in the art that solutions of rocuronium should be stored in a refrigerator at 2 to 8° C. Compatible diluents include: 0.9% sodium chloride injection USP, 5% dextrose injection USP, 5% dextrose and 0.9% sodium chloride injection USP, and sterile water for injection USP, and lactated ringer's injection USP.

It is well known in the art that solutions of vecuronium should be stored in a refrigerator below 30° C., and discarded after 24 hrs.

It is well known in the art that solutions of cisatracurium besylate should be stored in a refrigerator at 2 to 8° C., as well as protected from light.

It is well known in the art that solutions of pancuronium should be stored in a refrigerator at 2 to 8° C. In common practice a solution of pancuronium bromide is delivered from the manufacturer at the most commonly utilized concentration of 1 mg/ml. Alternately, a stock solution is diluted with bacteriostatic water for injection, or a compatible diluent to obtain a solution containing 1 mg/ml pancuronium bromide. Compatible diluents include: 0.9% sodium chloride injection USP, 5% dextrose injection USP, 5% dextrose and 0.9% sodium chloride injection USP, and sterile water for injection USP, and lactated ringer's injection USP.

It is well known in the art that solutions of succinylcholine should be stored in a refrigerator at 2 to 8° C. Solutions of succinylcholine containing a preservative such as methylparaben may be stored at room temperature for up to 14 days.

### Methods of Use

The delivery of colored solutions of atracurium besylate is parenteral, with intravenous being especially preferred. The minimal dosage of a colored solution of atracurium besylate is the lowest dosage which elicits a muscle relaxant response in the mammal. For example, atracurium besylate solutions can be administered at dosages from 0.1 mg/kg to 1 mg/kg. Preferred doses are 0.1 mg/kg to 0.5 mg/kg as required, an example in preparation for endotracheal intubation being 0.4 mg/kg. Maximal dosage for a mammal is the highest dosage which elicits muscle relaxation which does not cause undesirable or intolerable side effects such as changes in heart rate or blood pressure. In any event, the practitioner is guided by skill and knowledge in the field, and the present invention includes without limitation dosages which are effective to achieve the described effect in the mammal.

The delivery of colored solutions of rocuronium is parenteral, with intravenous being especially preferred. The minimal dosage of a colored solution of rocuronium is the lowest dosage which elicits a muscle relaxant response in the mammal. For example, rocuronium solutions can be administered at a dosage of from 0.1 mg/kg to 2 mg/kg. Preferred doses are 0.2 mg/kg to 1.2 mg/kg as required, an example for endotracheal intubation being 0.8 mg/kg. Maximal dosage for a mammal is the highest dosage which elicits muscle relaxation which does not cause undesirable or intolerable side effects such as allergic reactions. In any event, the practitioner is guided by skill and knowledge in the field, and the present invention includes without limitation dosages which are effective to achieve the described effect in the mammal.

The delivery of colored solutions of vecuronium is parenteral, with intravenous being especially preferred. The minimal dosage of a colored solution of vecuronium is the lowest dosage which elicits a muscle relaxant response in the mammal. For example, vecuronium solutions can be administered at dosages from 0.01 mg/kg to 0.3 mg/kg. Preferred doses are 0.04 mg/kg to 0.1 mg/kg as required, an example for endotracheal intubation being 0.08 mg/kg. Vecuronium bromide may also be given by continuous infusion, a typical dose being about 0.001 mg/kg/minute. Maximal dosage for a mammal is the highest dosage which elicits muscle relaxation which does not cause undesirable or intolerable side effects such as allergic reactions. In any event, the practitioner is guided by skill and knowledge in the field, and the present invention includes without limitation dosages which are effective to achieve the described effect in the mammal.

The delivery of colored solutions of cisatracurium besylate is parenteral, with intravenous being especially preferred. The minimal dosage of a colored solution of cisatracurium besylate is the lowest dosage which elicits a muscle relaxant response in the mammal. For example, cisatracurium besylate solutions can be administered at dosages from 0.01 mg/kg to 1 mg/kg. Preferred doses are 0.05 mg/kg to 0.15 mg/kg as required, an example for endotracheal intubation being 0.1 mg/kg. Maximal dosage for a mammal is the highest dosage which elicits muscle relaxation which does not cause undesirable or intolerable side effects such as allergic reactions. In any event, the practitioner is guided by skill and knowledge in the field, and the present invention includes without limitation dosages which are effective to achieve the described effect in the mammal.

The delivery of colored solutions of pancuronium is parenteral, with intravenous being especially preferred. The minimal dosage of a colored solution of pancuronium is the lowest dosage which elicits a muscle relaxant response in the mammal. For example, pancuronium solutions can be administered at a dosage ranging from 0.02 mg/kg to 0.5 mg/kg. Preferred doses are 0.04 mg/kg to 0.1 mg/kg as required, an example for endotracheal intubation being 0.1 mg/kg. Maximal dosage for a mammal is the highest dosage which elicits muscle relaxation which does not cause undesirable or intolerable side effects such as increased heart rate or blood pressure. In any event, the practitioner is guided by skill and knowledge in the field, and the present invention includes without limitation dosages which are effective to achieve the described effect in the mammal.

The delivery of colored solutions of succinylcholine is parenteral, preferred methods being intravenous and intramuscular, with intravenous being especially preferred. The minimal dosage of a colored solution of succinylcholine is the lowest dosage which elicits a muscle relaxant response in the mammal. For example, succinylcholine solutions can be administered at dosages from 0.25 mg/kg to 2 mg/kg. Preferred doses are 1 mg/kg to 1.5 mg/kg as required, an example being 1 mg/kg. Maximal dosage for a mammal is the highest dosage which elicits muscle relaxation which does not cause undesirable or intolerable side effects such as allergic reaction, hyperkalemia, or cardiac dysrthymias. In any event, the practitioner is guided by skill and knowledge in the field, and the present invention includes without limitation dosages which are effective to achieve the described effect in the mammal.

The delivery of colored solutions of gantacurium is parenteral, with intravenous being especially preferred. The minimal dosage of a colored solution of pancuronium is the lowest dosage which elicits a muscle relaxant response in the mammal. For example, gantacurium solutions can be administered at a dosage ranging from 0.02 mg/kg to 0.5 mg/kg. Preferred doses are 0.04 mg/kg to 0.1 mg/kg as required, an example for endotracheal intubation being 0.1 mg/kg. Maximal dosage for a mammal is the highest dosage which elicits muscle relaxation which does not cause undesirable or intolerable side effects such as hypotension. In any event, the practitioner is guided by skill and knowledge in the field, and the present invention includes without limitation dosages which are effective to achieve the described effect in the mammal.

The delivery of colored solutions of mivacurium is parenteral, with intravenous being especially preferred. The minimal dosage of a colored solution of pancuronium is the lowest dosage which elicits a muscle relaxant response in the mammal. For example, mivacurium solutions can be administered at a dosage ranging from 0.02 mg/kg to 0.5 mg/kg. Preferred doses are 0.04 mg/kg to 0.1 mg/kg as required, an example for endotracheal intubation being 0.1 mg/kg. Maximal dosage for a mammal is the highest dosage which elicits muscle relaxation which does not cause undesirable or intolerable side effects such as hypotension. In any event, the practitioner is guided by skill and knowledge in the field, and the present invention includes without limitation dosages which are effective to achieve the described effect in the mammal.

The delivery of colored solutions of diazepam is parenteral, preferred methods being intravenous and intramuscular, with intravenous being especially preferred. The minimal dosage of a colored solution of diazepam is the lowest dosage which elicits the desired effect in a mammal. For example, diazepam solutions can be administered at dosages from 0.5 mg/kg to 20 mg/kg. Preferred doses are 1 mg/kg to 10 mg/kg as required, an example being 2 mg/kg. For example, to induce anterograde amnesia, colored solutions of diazepam may be administered at dosages ranging from 5 mg to 15 mg. For example, to relieve acute anxiety, colored solutions of diazepam may be administered at dosages ranging from 2 mg to 5 mg for moderate anxiety and 5 mg to 10 mg for severe anxiety. For example, to relieve status epilepticus, colored solutions of diazepam may be administered at dosages ranging from 5 mg to 10 mg. For example, to relieve acute agitation, tremor, impending or acute delirium tremens, and hallucinations due to acute alcohol withdrawal, colored solutions of diazepam may be administered in a 10 mg dose initially, then 5 mg to 10 mg in 3 to 4 hours, if necessary. For example, to relieve muscle spasms associated with local pathology, cerebral palsy, athetosis, stiff-man syndrome, or tetanus, colored solutions of diazepam may be administered at dosages ranging from 5 mg to 10 mg initially, then 5 mg to 10 mg in 3 to 4 hours, if necessary. For tetanus, larger doses may be required. Maximal dosage for a mammal is the highest dosage which elicits the desired effect which does not cause undesirable or intolerable side effects such as respiratory arrest. In any event, the practitioner is guided by skill and knowledge in the field, and the present invention includes without limitation dosages which are effective to achieve the described effect in the mammal.

The delivery of colored solutions of lorazepam is parenteral, preferred methods being intravenous and intramuscular, with intravenous being especially preferred. The minimal dosage of a colored solution of lorazepam is the lowest dosage which relieves anxiety, controls seizures, or induces anterograde amnesia in a mammal. For example, lorazepam solutions can be administered intravenously at dosages from 0.002 mg/kg to 0.05 mg/kg with a total dose not typically exceeding a total of 4 mg. Preferred doses are 0.002 mg/kg to 0.006 mg/kg titrated as required, an example being an initial dose of 0.4 mg. Maximal dosage for a mammal is the highest dosage which relieves anxiety, controls seizures, or induces anterograde amnesia which does not cause undesirable or intolerable side effects such as respiratory arrest. In any event, the practitioner is guided by skill and knowledge in the field, and the present invention includes without limitation dosages which are effective to achieve the described effect in the mammal.

The delivery of colored solutions of midazolam hydrochloride is parenteral, preferred methods being intravenous and intramuscular, with intravenous being especially preferred.

The minimal dosage of a colored solution of midazolam hydrochloride is the lowest dosage which elicits sedation, in a mammal. For example, midazolam hydrochloride solutions can be administered intravenously and titrated to effect starting with dosages of 0.25 mg up to 10 mg. Preferred intravenous doses are in the range of 0.5 mg to 2 mg as required, an example being 1 mg at a time. General anesthesia may be induced by the administration of midazolam at a dose ranging from 0.05 mg/kg to 0.5 mg/kg, most preferably from 0.1 mg/kg to 0.2 mg/kg. The onset of unconsciousness may be facilitated by a small dose of an opioid preceding the dose of midazolam. Midazolam may also be used to provide sedation for intubated patients in the setting of an intensive care unit by using a maintenance infusion of 1 to 7 mg/hour titrated to effect. Maximal dosage for a mammal is the highest dosage which elicits sedation, anesthesia, or anterograde amnesia which does not cause undesirable or intolerable side effects such as respiratory arrest. In any event, the practitioner is guided by skill and knowledge in the field, and the present invention includes without limitation dosages which are effective to achieve the described effect in the mammal.

The delivery of colored solutions of etomidate is parenteral, with intravenous being especially preferred. The minimal dosage of a colored solution of etomidate is the lowest dosage which induces general anesthesia in the mammal. For example, etomidate solutions can be administered at dosages from 0.15 mg/kg to 0.5 mg/kg. Preferred doses for the induction of general anesthesia are in the range of 0.3 mg/kg. Another example of the minimal dosage of a colored solution of etomidate is the lowest dosage which maintains general anesthesia in the mammal. For example, etomidate solutions can be administered as an infusion at dosages from 10 to 100 mcg/kg/min. Etomidate used for maintenance of general anesthesia may be combined with an inhaled agent such as nitrous oxide, a halogenated methyl ethyl ether (isoflurane), or any of a number of inhaled volatile agents, (desflurane, halothane, sevoflurane). A narcotic infusion is often required in conjunction with etomidate and these agents are titrated in order to optimize the clinical efficacy of the medications. Maximal dosage for a mammal is the highest dosage which induces general anesthesia which does not cause undesirable or intolerable side effects such as transient venous pain, and transient skeletal muscle movements. In any event, the practitioner is guided by skill and knowledge in the field, and the present invention includes without limitation dosages which are effective to achieve the described effect in the mammal.

The delivery of colored solutions or emulsions of propofol is parenteral, with intravenous being especially preferred. An example of a minimal dosage of a colored solution or emulsion of propofol is the lowest dosage which induces general anesthesia in the mammal. For example, propofol solutions or emulsions can be used as a bolus at dosages from 0.3 mg/kg to 30 mg/kg. Preferred doses are 1 mg/kg to 5 mg/kg as required, an example being 2 mg/kg. Another example of a minimal dosage of a colored solution or emulsion of propofol is the lowest dosage which maintains general anesthesia in the mammal. The maintenance of general anesthesia may be accomplished using a continuous infusion of propofol at a dose appropriate for achieving hypnosis (50-200 mcg/kg/min). Alternatively, the general anesthetic may be maintained with an inhaled agent such as a halogenated methyl ethyl ether (isoflurane), or any of a number of inhaled volatile agents, (desflurane, halothane, sevoflurane). These halogenated agents are often used in combination with nitrous oxide, infusions of narcotics, or infusions of propofol. When propofol is used as an infusion along with either an inhaled agent or tandem infusion, the agents are titrated in order to accomplish the goals of the anesthetic. Another example of a minimal dosage of a colored solution or emulsion of propofol is the lowest dosage which induces sedation in the mammal. For example, propofol solutions or emulsions can be titrated for the purposes of sedation utilizing an infusion pump, at dosages ranging from 25 micrograms/kg/min to 100 micrograms/kg/min. For those patients requiring hypnosis, infusion doses ranging from 50 micrograms/kg/min to 200 micrograms/kg/min are appropriate. Maximal dosage for a mammal is the highest dosage which induces general anesthesia which does not cause undesirable or intolerable side effects such as transient venous pain, and hypotension. In any event, the practitioner is guided by skill and knowledge in the field, and the present invention includes without limitation dosages which are effective to achieve the described effect in the mammal.

The delivery of colored solutions of fospropofol disodium is parenteral, with intravenous being especially preferred. An example of a minimal dosage of a colored solution of fospropofol disodium is the lowest dosage which induces general anesthesia in the mammal. For example, fospropofol disodium solutions can be used as a bolus at dosages from 0.3 mg/kg to 30 mg/kg. Preferred doses are 1 mg/kg to 5 mg/kg as required, an example being 2 mg/kg. Another example of a minimal dosage of a colored solution of fospropofol disodium is the lowest dosage which maintains general anesthesia in the mammal. The maintenance of general anesthesia may be accomplished using a continuous infusion of fospropofol disodium at a dose appropriate for achieving hypnosis (50-200 mcg/kg/min). Alternatively, the general anesthetic may be maintained with an inhaled agent such as a halogenated methyl ethyl ether (isoflurane), or any of a number of inhaled volatile agents, (desflurane, halothane, sevoflurane). These halogenated agents are often used in combination with nitrous oxide, infusions of narcotics, or infusions of fospropofol disodium. When fospropofol disodium is used as an infusion along with either an inhaled agent or tandem infusion, the agents are titrated in order to accomplish the goals of the anesthetic. Another example of a minimal dosage of a colored solution of fospropofol disodium is the lowest dosage which induces sedation in the mammal. For example, fospropofol disodium solutions can be titrated for the purposes of sedation utilizing an infusion pump, at dosages ranging from 25 micrograms/kg/min to 100 micrograms/kg/min. For those patients requiring hypnosis, infusion doses ranging from 50 micrograms/kg/min to 200 micrograms/kg/min are appropriate. Maximal dosage for a mammal is the highest dosage which induces general anesthesia which does not cause undesirable or intolerable side effects such as transient venous pain, and hypotension. In any event, the practitioner is guided by skill and knowledge in the field, and the present invention includes without limitation dosages which are effective to achieve the described effect in the mammal.

The delivery of colored solutions of ketamine hydrochloride is parenteral, with intravenous being especially preferred. The minimal dosage of a colored solution of ketamine hydrochloride is the lowest dosage which induces general anesthesia in the mammal. For example, intravenous ketamine hydrochloride solutions can be administered at dosages from 0.02 mg/kg to 10 mg/kg. Preferred intravenous doses are 0.5 mg/kg to 4 mg/kg as required, a preferred example for the induction of anesthesia being 2 mg/kg. Colored solutions of ketamine hydrochloride may also be used in conjunction with diazepam for the induction of general anesthesia such as for example 5 to 15 mg of diazepam. Another example of the minimal dosage of a colored solution of ketamine hydrochloride is the lowest dosage which provides analgesia in the mammal. For example, intravenous ketamine hydrochloride solutions can be administered at dosages from 0.002 mg/kg/minute to 0.02 mg/kg/minute with a preferred example being 0.004 mg/kg/minute. Colored solutions of ketamine hydrochloride may also be used in conjunction with diazepam for the maintenance of general anesthesia, such as for example 2 to 5 mg diazepam. Maximal dosage for a mammal is the highest dosage which induces general anesthesia which does not cause undesirable or intolerable side effects such as emergence delirium, increased heart rate, contractility, and intracranial pressure. In any event, the practitioner is guided by skill and knowledge in the field, and the present invention includes without limitation dosages which are effective to achieve the described effect in the mammal.

The delivery of colored solutions of atropine is parenteral, with intravenous being especially preferred. The minimal dosage of a colored solution of atropine is the lowest dosage which induces an anticholinergic effect in the mammal. For example, atropine solutions can be administered at dosages from 0.01 mg to 200 mg, 0.1 mg to 1 mg as required, an example being 0.5 mg. Maximal dosage for a mammal is the highest dosage which induces an anticholinergic effect which does not cause undesirable or intolerable side effects such as tachycardia, central nervous system toxicity, mydriasis, cycloplegia, hyperthermia, and excessive drying of airway secretions. In any event, the practitioner is guided by skill and knowledge in the field, and the present invention includes without limitation dosages which are effective to achieve the described effect in the mammal.

The delivery of colored solutions of glycopyrrolate is parenteral, with intravenous being especially preferred. The minimal dosage of a colored solution of glycopyrrolate is the lowest dosage which induces an anticholinergic effect in the mammal. For example, glycopyrrolate solutions can be administered at dosages from 0.002 mg/kg up to 1 mg total. When used for purposes of premedication, doses ranging from 0.005 to 0.01 mg/kg are often reasonable; when used in conjunction with an anticholinesterase for the reversal of pharmacologic nondepolarizing neuromuscular blockade, doses in the range of 0.01 mg/kg are typical. Maximal dosage for a mammal is the highest dosage which induces an anticholinergic effect which does not cause undesirable or intolerable side effects such as tachycardia, mydriasis, cycloplegia, hyperthermia, and excessive drying of airway secretions. In any event, the practitioner is guided by skill and knowledge in the field, and the present invention includes without limitation dosages which are effective to achieve the described effect in the mammal.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-285. (canceled)

286. A colored injectable pharmaceutical, wherein said color is derived from a dye comprising fluorescein.

287. The colored injectable pharmaceutical of claim 286, wherein said color is derived from a dye comprising fluorescein and methylene blue.

288. The colored injectable pharmaceutical of claim 286, wherein said color is derived from a dye comprising fluorescein and indigo carmine.

289. The colored injectable pharmaceutical of claim 286, wherein said pharmaceutical is a muscle relaxant, and wherein said color is derived from a dye comprising fluorescein in the concentration of 7 mg/mL to 250 mg/mL, and wherein the color is bright orange.

290. The colored injectable pharmaceutical of claim 286, wherein said pharmaceutical is a hypnotic, and wherein said color is derived from a dye comprising fluorescein in the concentration of 1 mg/mL to 10 mg/mL, and wherein the color is orange.

300. The colored injectable hypnotic of claim 290, wherein said color is derived from a dye comprising fluorescein in the concentration of 1.5 mg/mL to 2.5 mg/mL, and wherein the color is orange.

301. The colored injectable pharmaceutical of claim 286, wherein said pharmaceutical is a induction agent, and wherein said color is derived from a dye comprising fluorescein in the concentration of 0.001 mg/mL to 2.5 mg/mL, and wherein the color is yellow.

302. The colored injectable pharmaceutical of claim 286, wherein said pharmaceutical is an anticholinergic, wherein said color is derived from a dye comprising fluorescein in the concentration of 0.001 mg/mL to 0.1 mg/mL and methylene blue in the concentration of 0.001 mg/mL to 0.05 mg/mL, and wherein the color is green.

303. The colored injectable pharmaceutical of claim 286, wherein said pharmaceutical is an anticholinergic, wherein said color is derived from a dye comprising fluorescein in the concentration of 0.001 mg/mL to 0.4 mg/mL and indigo carmine in the concentration of 0.001 mg/mL to 0.05 mg/mL, and wherein the color is green.

304. A method of preparing color coded injectable pharmaceuticals, wherein different colors are created by varying the concentration of fluorescein.

305. The method of claim 304, wherein the colors yellow, orange, and bright orange are created by varying the concentration of fluorescein.

306. The pharmaceutical composition of claim 286, wherein the pharmaceutical is atracurium besylate and wherein the color is bright orange.

307. The pharmaceutical composition of claim 286, wherein the pharmaceutical is rocuronium and wherein the color is bright orange.

308. The pharmaceutical composition of claim 286, wherein the pharmaceutical is vecuronium and wherein said color is bright orange.

309. The pharmaceutical composition of claim 286, wherein the pharmaceutical is cisatracurium besylate and wherein the color is bright orange.

310. The pharmaceutical composition of claim 286, wherein the pharmaceutical is pancuronium and wherein the color is bright orange.

311. The pharmaceutical composition of claim 286, wherein the pharmaceutical is succinylcholine and wherein the color is bright orange.

312. The pharmaceutical composition of claim 286, wherein the pharmaceutical is lorazepam and wherein said color is orange.

313. The pharmaceutical composition of claim 286, wherein the pharmaceutical is midazolam hydrochloride and wherein said color is orange.

314. The pharmaceutical composition of claim 286, wherein the pharmaceutical is diazepam and wherein said composition is a solution and said color is orange.

315. The pharmaceutical composition of claim 286, wherein the pharmaceutical is etomidate and wherein the color is yellow.

316. The pharmaceutical composition of claim 286, wherein the pharmaceutical is propofol and wherein the color is yellow.

317. The pharmaceutical composition of claim 286, wherein the pharmaceutical is fospropofol disodium and wherein the color is yellow.

318. The pharmaceutical composition of claim 286, wherein the pharmaceutical is fospropofol disodium and wherein the color is orange.

319. The pharmaceutical composition of claim 286, wherein the pharmaceutical is ketamine hydrochloride and wherein the color is yellow.

320. The pharmaceutical composition of claim 286, wherein the pharmaceutical is atropine, and wherein the color is green derived from fluorescein and methylene blue.

321. The pharmaceutical composition of claim 286, wherein the pharmaceutical is atropine, and wherein the color is green derived from fluorescein and indigo carmine.

322. The pharmaceutical composition of claim 286, wherein the pharmaceutical is glycopyrrolate and wherein the color is green derived from fluorescein and methylene blue.

323. The pharmaceutical composition of claim 286, wherein the pharmaceutical is glycopyrrolate and wherein the color is green derived from fluorescein and indigo carmine.

324. The pharmaceutical composition of claim 286, wherein the pharmaceutical is gantacurium and wherein the color is bright orange.

325. The pharmaceutical composition of claim 286, wherein the pharmaceutical is mivacurium and wherein the color is bright orange.
